(19) United States
(12) Patent Application Publication
(10) Pub. No.: US 2022/0313782 A1
(43) Pub. Date: Oct. 6, 2022

(54) METHODS AND COMPOSITIONS INVOLVING TERT ACTIVATING THERAPIES

(71) Applicant: BOARD OF REGENTS, THE UNIVERSITY OF TEXAS SYSTEM, Austin, TX (US)

(72) Inventors: Ronald A. DEPINHO, Houston, TX (US); Yaoqi Alan WANG, Houston, TX (US); Hong Seok SHIM, Houston, TX (US); James W. HORNER, Houston, TX (US)

(73) Assignee: BOARD OF REGENTS, THE UNIVERSITY OF TEXAS SYSTEM, Austin, TX (US)

(21) Appl. No.: 17/608,025

(22) PCT Filed: Apr. 30, 2020
(86) PCT No.: PCT/US2020/030699

§ 371 Date: Nov. 1, 2021

Related U.S. Application Data
(60) Provisional application No. 62/842,323, filed on May 2, 2019.

(51) Int. Cl. A61K 38/17; A61K 38/10; A61K 38/45; A61K 35/28; A61K 35/33

(52) U.S. Cl. CPC A61K 38/1716; A61K 38/10; A61K 38/45; A61K 35/28; A61K 35/33

(57) ABSTRACT

The disclosure provides for methods and compositions for treating a premature aging disorder or neurodegenerative disorder, particularly neurodegenerative disorders associated with amyloid deposition and neuronal death, such as Alzheimer's disease. Accordingly, aspects of the disclosure relate to a method for treating a premature aging disorder in a subject in need thereof, comprising administering a TERT activating therapy to the subject. Further aspects relate to a method for treating a neurodegenerative disorder in a subject comprising administering a TERT activating therapy to the subject.

### BACKGROUND OF THE INVENTION

This application claims priority to U.S. Provisional Application No. 62/842,323, filed on May 2, 2019, the entirety of which is incorporated herein by reference.

This invention was made with government support under grant number CA084628 awarded by the National Institutes of Health. The government has certain rights in the invention.

### I. Field of the Invention

This invention relates to the field of medicine. Specifically, this invention provides methods and compositions for treating Alzheimer's disease.

### II. Background

Alzheimer's disease (AD) is a progressive and degenerative disease. It is characterized by increased levels of pro-inflammatory cytokines and build-up of toxic .beta.-amyloid depositions, especially in the hippocampus, that gradually destroys memory and the ability to learn. Despite medical advances, there are no definitive therapies for AD. FDA-approved drugs only temporarily slow the worsening of symptoms, and only in about half of the patients who take these medications. This translates into combined direct and indirect costs of AD and other dementias to Medicare, Medicaid, and businesses in excess of $148 billion each year.

Current approved drug treatments for cognitive symptoms of AD include cholinesterase inhibitors, while ‘off-label’ treatment of behavioral symptoms of AD include antidepressant drugs; both of these drug classes, in addition to increasing neurotransmitter availability, elicit unfavorable side effects and inhibit the production of tumor necrosis factor. The problems of high costs, unfavorable side effects, and limited efficacy need to be resolved in treatments of AD. The need exists for therapies that address issues related to AD, such as, high costs, high occurrence of unfavorable side effects, and existing limitations in efficacious treatment of AD.

### SUMMARY OF THE INVENTION

The disclosure provides for methods and compositions for treating a premature aging disorder or neurodegenerative disorder, particularly those associated with amyloid deposition and neuronal death, such as Alzheimer's disease. Accordingly, aspects of the disclosure relate to a method for treating a premature aging disorder in a subject in need thereof, comprising administering a TERT activating therapy to the subject. Further aspects relate to a method for treating a neurodegenerative disorder in a subject comprising administering a TERT activating therapy to the subject. Further aspects relate to a method for generating new neurons in a subject in need thereof, comprising administering a TERT activating therapy to the subject. Further aspects relate to a method for reducing amyloid-β peptide in a subject in need thereof, comprising administering a TERT activating therapy to the subject. Yet further aspects relate to a composition comprising a nanovesicle comprising a TERT polypeptide and/or a nucleic acid encoding a TERT polypeptide. A TERT activating therapy refers to a therapy that may do one or more of increase expression of the endogenous TERT protein, increase concentration of the TERT protein in a cell, increases the activity of the TERT protein (ether endogenously added TERT protein or exogenously added TERT protein), and stabilize the TERT protein and/or mRNA.

In some embodiments, the premature aging disorder comprises Hutchinson—Gilford progeria syndrome (HGPS), Nestor—Guillermo progeria syndrome, Werner syndrome, Cockayne syndrome, Bloom syndrome, Xeroderma pigmentosum, Ataxia telangiectasia, Trichothiodystrophy, Dyskeratosis congenital, or Mosaic variegated aneuploidy syndrome. In some embodiments, the neurodegenerative disorder comprises Alzheimer's disease. In some embodiments, the premature aging disorder excludes Hutchinson—Gilford progeria syndrome (HGPS), Néstor-Guillermo progeria syndrome, Werner syndrome, Cockayne syndrome, Bloom syndrome, Xeroderma pigmentosum, Ataxia telangiectasia, Trichothiodystrophy, Dyskeratosis congenital, or Mosaic variegated aneuploidy syndrome. In some embodiments, the neurodegenerative disorder excludes Alzheimer's disease. In some embodiments, Alzheimer's disease comprises or is early onset Alzheimer's disease. In some embodiments, Alzheimer's disease comprises or is late onset Alzheimer's disease. In some embodiments, either early onset Alzheimer's disease or late onset Alzheimer's disease is excluded. In some embodiments, the neurodegenerative disorder comprises a neurodegenerative disorder associated with amyloid deposition. In some embodiments, neurodegenerative is defined as a disease comprising degeneration and/or death of nerve cells. In some embodiments, the neurodegenerative disorder is one that causes neuronal cell death. In some embodiments, treating comprises increasing dendritic spine formation. In some embodiments, the increase of dendritic spine formation is in cortical neurons. In some embodiments, treating comprises increasing or enhancing neural networks. In some embodiments, treating comprises enhancing or increasing synaptic pathway activation, which promotes molecular chaperon expression and reduces the expression of AD risk genes. In some embodiments, treating comprises reducing amyloid plaques. In some embodiments, the subject has been diagnosed with the disorder. In some embodiments, the subject has previously been treated for the disorder. In some embodiments, the subject has been determined to be non-responsive to the previous therapy.

In some embodiments, the subject has not been previously treated for the disorder. In some embodiments, the subject is a human. In some embodiments, the subject is less than 50 years old. In some embodiments, the subject is less than or more than 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 years old (or any range derivable therein).

In some embodiments, the method further comprises administration of an additional therapy. In some embodiments, the additional therapy comprises a cholinesterase inhibitor such as donepezil, galantamine, or rivastigmine. In some embodiments, the additional therapy comprises memantine. In some embodiments, the methods and compositions of the disclosure exclude one or more of donepezil, galantamine, rivastigmine, or memantine.

In some embodiments, the TERT activating therapy comprises delivery of nucleic acids encoding a TERT polypeptide. In some embodiments, the TERT nucleic acids comprise a nucleic acid of SEQ ID NO:1, 3, 5, 7, or 9, or fragments thereof, or a nucleic acid with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity to one of SEQ ID NO:1, 3, 5, 7, or 9, or a fragment thereof In some embodiments, the TERT activating therapy comprises a DNA or RNA encoding for a TERT polypeptide to the subject. In some embodiments, the TERT activating therapy comprises a TERT polypeptide. In some embodiments, the TERT polypeptide comprises a polypeptide of SEQ ID NO:2, 4, 6, 8, or 10, or fragments thereof, or a nucleic acid with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity to one of SEQ ID NO: 2, 4, 6, 8, or 10, or fragments thereof. In some embodiments, the TERT activating therapy comprises a catalytically inactive TERT polypeptide, such as a TERT polypeptide capable of transactivation of genes but lacking

Telomerase Reverse Transcriptase activity. In some embodiments the TERT polypeptide comprises a D712A mutation. In some embodiments, the TERT polypeptide does not have a D712A mutation.

In some embodiments, the TERT activating therapy comprises a nanovesicle comprising a TERT polypeptide or a nucleic acid encoding for a TERT polypeptide. In some embodiments, the nanovesicle comprises an exosome. In some embodiments, the nanovesicle is 10-1000 nm in diameter. In some embodiments, the nanovesicle is at least or at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nm in diameter (or any derivable range therein). In some embodiments, the nanovesicle comprises CD47. In some embodiments, the nanovesicle comprises expression of CD47 on the surface and/or within the membrane of the nanovesicle. In some embodiments, the nanovesicle comprises a rabies virus glycoprotein peptide. Exemplary rabies virus glycoprotein peptides useful in embodiments of the disclosure include the following:

| | SEQ |
|---|---|
| Rabies virus glycoprotein peptide sequence | ID NO: |
| YTIWMPENPRPGTPCDIFTNSRGKRASNG | 11 |
| YTIWMPENPRPGTPCDIFTNSRGKRASNGC | 12 |
| YTIWMPENPRPGTPCDIFTNSRGKRASNGGGGGC | 13 |
| YTIWMPENPRPGTPCDIFTNSRGKRASNGGGGG9dR | 14 |

dR = D-arginine

The rabies virus glycoprotein peptide may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 (or any derivable range therein) or more variant amino acids or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 or more contiguous amino acids, or any range derivable therein, of SEQ ID Nos:11-14.

The rabies virus glycoprotein peptide may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 or more contiguous amino acids, or any range derivable therein, of SEQ ID NOS:11-14.

In some embodiments, the rabies virus glycoprotein peptide comprises amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 (or any derivable range therein) of SEQ ID NOs:11-14.

The rabies virus glycoprotein peptide may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 substitutions.

In some embodiments, the compositions and methods exclude exosomes or nanovesicles as a delivery method for a TERT

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42.

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 amino acids (or any derivable range therein).

In some embodiments, the TERT activating therapy comprises modulation of a histone H3K9 methyltransferases (HMTs). In some embodiments, the modulation comprises repression of the HMT gene or protein. In some embodiments, the repression comprises genetic silencing of one or more HMT genes. Methods for genetic silencing are known in the art. For example methods such as homology directed repair and gene editing may be used to mutate one or more HMT genes in cells in the subject, such as in neuronal cells or support cells. In some embodiments, gene editing techniques such as CRISPR, are used to decrease the expression of one or more HMTs in a subject. In some embodiments, the one or more HMT genes comprise one or more of SUV39H1/KMT1A, SUV39H2/KMT1B, SETDB1/KMT1E, SETDB2/KMT1F, PRDM2, G9A/KMT1C, GLP/KMT1D, EHMT1, and RIZ1/KMT8. In some embodiments, one or more of SUV39H1/KMT1A, SUV39H2/KMT1B, SETDB1/KMT1E, SETDB2/KMT1F, PRDM2, G9A/KMT1C, GLP/KMT1D, EHMT1, and RIZ1/KMT8 is excluded as an HMT embodiment. In some embodiments, the TERT activating therapy comprises a HMT inhibitor. In some embodiments, the HMT inhibitor comprises one or more of Chaetocin, BIX-01294, BIX-01338, UNC0638, and BRD4770. In some embodiments, one or more of Chaetocin, BIX-01294, BIX-01338, UNC0638, and BRD4770 is excluded. In some embodiments, the TERT activating therapy comprises Chaetocin. In some embodiments, the TERT activating therapy comprises administration of a histone H3K9 demethylase (HDM) polypeptide or a nucleic acid encoding a HDM. In some embodiments, the HDM polypeptide comprises a polypeptide with demethylase activity. In some embodiments, the HDM polypeptide comprises a polypeptide from one or more of KDM1A/LSD1, KDM3A/JHDM2A, KDM3B/JHDM2B, KDM4A/JHDM3A, KDM4B/JMJD2B, KDM4C/JMJD2C, KDM4D/JMJD2D, KDM7/JHDM1D, and PHF8. In some embodiments, one or more of KDM1A/LSD1, KDM3A/JHDM2A, KDM3B/JHDM2B, KDM4A/JHDM3A, KDM4B/JMJD2B, KDM4C/JMJD2C, KDM4D/JMJD2D, KDM7/JHDM1D, and PHF8 is excluded as a HDM embodiment.

Other embodiments of the rabies virus glycoprotein are further described in Oswald et al., Mol. Pharmaceutics, 2017, 14 (7), pp 2177-2196, which is herein incorporated by reference.

In some embodiments, the nanovesicles are derived from fibroblasts or bone marrow dendritic cells. In some embodiments, the nanovesicles are derived from human cells. In some embodiments, the nanovesicles are derived from non-human cells.

In some embodiments, the TERT activating therapy is administered by intravenous injection. In some embodiments, the TERT activating therapy is administered systemically.

In some embodiments, the TERT activating therapy is administered by a route of administration described herein.

In some embodiments, treating comprises one or more of a reduction in amyloid-β peptide, an improvement in learning, an improvement in memory, and the generation of neurons. The reduction or improvement may be at least 10, 15, 20, 25, 30, 35, 40, 45, 50, 55, 60, 65, 70, 75, 80, 85, or 90%, or any range derivable therein.

In some embodiments, the TERT polypeptide comprises a polypeptide with telomerase activity. The terms “protein”, “polypeptide” and “peptide” are used interchangeably herein when referring to a gene product.

The terms “subject,” “mammal,” and “patient” are used interchangeably. In some embodiments, the subject is a mammal. In some embodiments, the subject is a human. In some embodiments, the subject is a mouse, rat, rabbit, dog, donkey, or a laboratory test animal such as fruit fly, zebrafish, etc.

In some embodiments, the subject has been previously treated for a disease or disorder. In some embodiments, the subject was resistant to the previous treatment. In some embodiments, the subject was determined to be a poor responder to the previous treatment.

It is contemplated that the methods and compositions include exclusion of any of the embodiments described herein.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-I. Tert is downregulated in two distinct mouse Alzheimer's neurons. (A)

Tert mRNA levels in the cortex of 3xTg-AD and wildtype control (B6129SF2/J) mice (n=4; 3-month-old). (B) Tert mRNA levels in the hippocampus of 5xFAD and wildtype littermate control mice (n=4; 2˜3-month-old). (C) Tert mRNA levels in primary cortical and hippocampal neurons isolated from 3xTg-AD and control mice at DIV 14 (n=3). (D) Tert mRNA levels in primary cortical and hippocampal neurons isolated from 5xFAD and control mice at DIV 14 (n=3). (E) Telomerase activity in hippocampal neurons isolated from 5xFAD and control mice (n=4; 2˜3-month-old). (F) Representative view of H3K9me3 repressive histone mark occupancy in Tert gene of 5xFAD and control mouse primary neurons at DIV 14. (G) mRNA levels of histone demethylase Kdm1a, Kdm4b, and Kdm4c genes in cortical and hippocampal neurons of 5xFAD and wildtype littermate control mice (n =4; 2˜3-month-old). (H) KDM1A immunostaining in the CA1 hippocampal subfield of 5xFAD and wildtype littermate control mice (2˜3-month-old). (I) Tert mRNA levels in the cortex and hippocampus of 5xFAD mice treated with chaetocin or BIX-01294.

FIG. 2A-C. Generation of Cre-inducible Tert knock-in mouse (R26-CAG-LSL-mTert-IRES-eGFP-pA). (A) Scheme of the construct used to introduce the CAG-LSL-mTert-IRES-eGFP-pA into Rosa26 locus. (B) Genotyping results of the original ES targeted lines carrying the R26-CAG-LSL-mTert-IRES-eGFP-pA alleles. (C) Representative photographs of chimeric mice obtained from targeted ES cells.

FIG. 3A-D. Tert activation alleviates amyloid pathology in the novel inducible TERT-AD mouse model. (A) Breeding strategy of R26-CAG-LSL-mTert with 3xTg-AD or 5xFAD and Camk2a-CreERT2 mice. (B) immunostaining in the CA1 hippocampal subfield of adult (8-month-old) control and Tert-activated R26-CAG-LSL-mTert; 3xTg-AD; Camk2a-CreERT2 mice. (C) Quantitative comparison of Aβ-immunoreactive pyramidal neurons in the CA1 regions (n =6 per group; 8-month-old). (D) Aβ immunostaining in the hippocampus of adult (7-month-old) control and Tert-activated R26-CAG-LSL-mTert; 5xFAD; Camk2a-CreERT2 mice.

FIG. 4A-F. Tert activation in AD neurons enhances various synaptic pathways which promote molecular chaperon expression and reduce the expression of AD risk genes. (A) mRNA levels of Tert and Terc in Tert-activated neurons of R26-CAG-LSL-mTert; 3xTg-AD; Camk2a-CreERT2 mouse brains. (B) Venn diagram showing intersections of upregulated biological processes based on the RNA-Seq results from Tert-activated cortical and hippocampal neurons of R26-CAG-LSL-mTert; 3xTg-AD; Camk2a-CreERT2 mice compared with control groups. (C) Top 5 overlapping pathways upregulated in both Tert-activated cortical and hippocampal neurons. (D) Gene Set Enrichment Analysis (GSEA) plots showing relative upregulation of synaptic signaling genes in Tert-activated cortical and hippocampal neurons by comparison with control neurons. (E,F) mRNA levels of App, ApoE, Hsp70-1 and Hsp70-2 genes with or without Tert induction.

FIG. 5A-C. Tert activation enhances spine morphology and neural networks in AD mouse model. (A) Representative images of Golgi-stained cortical neurons from aged (18 months) control and Tert-activated R26-CAG-LSL-mTert; 3xTg-AD; Camk2a-CreERT2 mice. (B) High magnification of dendritic spines in impregnated pyramidal cortical neurons of aged control and Tert-activated R26-CAG-LSL-mTert; 3xTg-AD; Camk2a-CreERT2 mice. (C) Quantification of dendritic spine density (n=20 dendrites per group from n=4 mice per group; 18-month-old). Student's t-test for two-group comparisons. ****P<0.0001; mean±s.e.m.

FIG. 6A-D. Activation of human TERT gene by HMT inhibitor and gene silencing in human AD neurons. (A) Representative view of H3K9me3 repressive histone mark occupancy in TERT gene of neurons differentiated from APP^{DP} patient- and non-demented control (NDC) individual-derived iPSCs. (B,C) TERT mRNA levels (B) and TERT protein levels (C) in chaetocin-treated human AD neurons. (D) Immunoblot of TERT protein levels in human AD neurons treated with siRNAs targeting histone methyltransferase genes, G9A or SETDB1.

FIG. 7A-E. TERT activation alleviates amyloid pathology in human AD neurons. (A) Cloning of Flag-tagged human TERT lentiviral expression construct. (B,C) Aβ₁₋₄₀ levels measured by sandwich ELISA in EGFP- or TERT-transduced neurons differentiated from APP^{DP} patient-derived iPSCs (n=3). (D) Immunoblots for the indicated endogenous proteins in EGFP- or TERT-transduced APP^{DP} neurons. A tubulin was used as a loading control. (E) Relative gene expression by quantitative RT-PCR in EGFP- or TERT-transduced APP′ neurons (n=4).

FIG. 8A-C. TERT's transactivation function is independent of its catalytic activity. (A) Schematic of catalytically inactive (CI) human TERT lentiviral expression construct. The white asterisk indicates the position of the single mutation D712A, which renders the protein catalytically inactive. (B) Immunoblots for the confirmation of Flag-tagged catalytically inactive TERT expression in HEK293 cells. (C) mRNA expression levels of each gene indicated. Transcript levels were normalized to HPRT1 mRNA.

FIG. 9A-D. Activation of neuronal TERT triggers the transactivation of specific genes associated with learning processes in AD neurons. (A) Venn diagram showing intersections of upregulated biological processes based on three independent RNA-Seq results from Tert-activated mouse cortical and hippocampal neurons of R26-CAG-LSL-mTert; 3xTg-AD; Camk2a-CreERT2 mice (n=4 for each group) and TERT-activated human APP^{DP} neurons (n=3) compared with each control group (all p<0.05). (B) List of 13 overlapping pathways upregulated in all Tert-activated mouse cortical and hippocampal AD neurons and TERT-activated human AD neurons. (C) GSEA plots showing relative upregulation of learning-related genes in Tert-activated cortical and hippocampal AD neurons and TERT-activated human AD neurons by comparison with each control group. (D) Escape latency of aged (22˜26 months) control and Tert-activated R26-CAG-LSL-mTert; 3xTg-AD; Camk2a-CreERT2 mice in the Barnes maze over training days (n=9 for each group). Two-way ANOVA with Sidak's multiple comparisons test; Student's t-test for two-group comparisons; One, two, three, or four symbols denote P<0.05, 0.01, 0.0005, 0.0001, respectively; mean±s.e.m.

FIG. 10A-C. Neuronal TERT physically interacts with β-catenin transcription factor and RNA polymerase II complex core component. (A) List of TERT-interacting proteins identified by mass spectrometry in human AD neurons. (B) RNA-Seq heat map of WNT signaling pathway genes in EGFP- and TERT-transduced human AD neurons (n=3) (C) Co-immunoprecipitation of endogenous β-Catenin (active), CREBBP, POLR2A, and TERT from human AD neurons.

FIG. 11A-C. A global enrichment of the association of TERT and β-Catenin/TCF7 on the genomic level. (A) ChIP-Seq density heat maps of TERT, β-Catenin (active) and TCF7 across the gene promoters of human AD neurons. (B) Chromatin-state maps showing β-Catenin (active), TCF7 and TERT binding peaks for the WNT9B, ATP1A3, HSPA12A, HSPA6, and MYC locus, as determined by ChIP-Seq. (C) Model for TERT action in transcriptional activation in AD neurons. In neuronal cells, TERT levels decrease at the early pathological stage of AD. Activation of neuronal TERT triggers the transcriptional induction of specific genes associated with synaptic signaling and learning processes in AD neurons, enabling to alleviate cognitive deficits.

### DETAILED DESCRIPTION OF THE INVENTION

The telomerase reverse transcriptase (TERT), a catalytic subunit of telomerase, has been reported to have a variety of beneficial and protective functions in multiple tissues of both rodent and human. However, the relationship between telomerase and amyloid pathology, a major hallmark of AD pathology, has not been investigated. AD is a progressive and adult-onset neurodegenerative disease. To test the impact of TERT reactivation in the brain, the inventors established an inducible telomerase activation AD (TERT-AD) mouse model to control temporal regulation of TERT gene expression. As shown in Example 1, telomerase activation can alleviate AD pathology in mouse and human AD models via its direct regulation of critical neuronal transcription networks affected in AD. Enhancing TERT level and activity in the brain provides for a therapeutic strategy for the prevention and treatment of Alzheimer's disease amyloid neuropathology.

### I. TERT Polypeptides

TERT, also known as CMM9, DKCA2, DKCB4, EST2, PFBMFT1, TCS1, TP2, TRT, hEST2, and hTRT in humans and EST2, TCS1, TP2, TR, and TRT in mice, is known in the art and exemplified by the following mRNA and protein sequences described herein.

For example, the human TERT gene is exemplified by Homo sapiens telomerase reverse transcriptase (TERT), transcript variant 2, mRNA (NCBI Reference Sequence: NM_001193376.1):

(SEQ ID NO: 1)
CAGGCAGCGCTGCGTCCTGCTGCGCACGTGGGAAGCCCTGGCCCCGGCCACCCC
CGCGATGCCGCGCGCTCCCCGCTGCCGAGCCGTGCGCTCCCTGCTGCGCAGCCAC
TACCGCGAGGTGCTGCCGCTGGCCACGTTCGTGCGGCGCCTGGGGCCCCAGGGC
TGGCGGCTGGTGCAGCGCGGGGACCCGGCGGCTTTCCGCGCGCTGGTGGCCCAG
TGCCTGGTGTGCGTGCCCTGGGACGCACGGCCGCCCCCCGCCGCCCCCTCCTTCC
GCCAGGTGTCCTGCCTGAAGGAGCTGGTGGCCCGAGTGCTGCAGAGGCTGTGCG
AGCGCGGCGCGAAGAACGTGCTGGCCTTCGGCTTCGCGCTGCTGGACGGGGCCC
GCGGGGGCCCCCCCGAGGCCTTCACCACCAGCGTGCGCAGCTACCTGCCCAACA
CGGTGACCGACGCACTGCGGGGGAGCGGGGCGTGGGGGCTGCTGCTGCGCCGCG
TGGGCGACGACGTGCTGGTTCACCTGCTGGCACGCTGCGCGCTCTTTGTGCTGGT
GGCTCCCAGCTGCGCCTACCAGGTGTGCGGGCCGCCGCTGTACCAGCTCGGCGCT
GCCACTCAGGCCCGGCCCCCGCCACACGCTAGTGGACCCCGAAGGCGTCTGGGA
TGCGAACGGGCCTGGAACCATAGCGTCAGGGAGGCCGGGGTCCCCCTGGGCCTG
CCAGCCCCGGGTGCGAGGAGGCGCGGGGGCAGTGCCAGCCGAAGTCTGCCGTTG
CCCAAGAGGCCCAGGCGTGGCGCTGCCCCTGAGCCGGAGCGGACGCCCGTTGGG
CAGGGGTCCTGGGCCCACCCGGGCAGGACGCGTGGACCGAGTGACCGTGGTTTC
TGTGTGGTGTCACCTGCCAGACCCGCCGAAGAAGCCACCTCTTTGGAGGGTGCGC
TCTCTGGCACGCGCCACTCCCACCCATCCGTGGGCCGCCAGCACCACGCGGGCCC
CCCATCCACATCGCGGCCACCACGTCCCTGGGACACGCCTTGTCCCCCGGTGTAC
GCCGAGACCAAGCACTTCCTCTACTCCTCAGGCGACAAGGAGCAGCTGCGGCCC
TCCTTCCTACTCAGCTCTCTGAGGCCCAGCCTGACTGGCGCTCGGAGGCTCGTGG
AGACCATCTTTCTGGGTTCCAGGCCCTGGATGCCAGGGACTCCCCGCAGGTTGCC
CCGCCTGCCCCAGCGCTACTGGCAAATGCGGCCCCTGTTTCTGGAGCTGCTTGGG
AACCACGCGCAGTGCCCCTACGGGGTGCTCCTCAAGACGCACTGCCCGCTGCGA
GCTGCGGTCACCCCAGCAGCCGGTGTCTGTGCCCGGGAGAAGCCCCAGGGCTCT
GTGGCGGCCCCCGAGGAGGAGGACACAGACCCCCGTCGCCTGGTGCAGCTGCTC
CGCCAGCACAGCAGCCCCTGGCAGGTGTACGGCTTCGTGCGGGCCTGCCTGCGC
CGGCTGGTGCCCCCAGGCCTCTGGGGCTCCAGGCACAACGAACGCCGCTTCCTCA
GGAACACCAAGAAGTTCATCTCCCTGGGGAAGCATGCCAAGCTCTCGCTGCAGG
AGCTGACGTGGAAGATGAGCGTGCGGGACTGCGCTTGGCTGCGCAGGAGCCCAG
GGGTTGGCTGTGTTCCGGCCGCAGAGCACCGTCTGCGTGAGGAGATCCTGGCCA
AGTTCCTGCACTGGCTGATGAGTGTGTACGTCGTCGAGCTGCTCAGGTCTTTCTTT
TATGTCACGGAGACCACGTTTCAAAAGAACAGGCTCTTTTTCTACCGGAAGAGTG
TCTGGAGCAAGTTGCAAAGCATTGGAATCAGACAGCACTTGAAGAGGGTGCAGC
TGCGGGAGCTGTCGGAAGCAGAGGTCAGGCAGCATCGGGAAGCCAGGCCCGCCC
TGCTGACGTCCAGACTCCGCTTCATCCCCAAGCCTGACGGGCTGCGGCCGATTGT
GAACATGGACTACGTCGTGGGAGCCAGAACGTTCCGCAGAGAAAAGAGGGCCG
AGCGTCTCACCTCGAGGGTGAAGGCACTGTTCAGCGTGCTCAACTACGAGCGGG
CGCGGCGCCCCGGCCTCCTGGGCGCCTCTGTGCTGGGCCTGGACGATATCCACAG
GGCCTGGCGCACCTTCGTGCTGCGTGTGCGGGCCCAGGACCCGCCGCCTGAGCTG
TACTTTGTCAAGGTGGATGTGACGGGCGCGTACGACACCATCCCCCAGGACAGG
CTCACGGAGGTCATCGCCAGCATCATCAAACCCCAGAACACGTACTGCGTGCGT
CGGTATGCCGTGGTCCAGAAGGCCGCCCATGGGCACGTCCGCAAGGCCTTCAAG
AGCCACGTCTCTACCTTGACAGACCTCCAGCCGTACATGCGACAGTTCGTGGCTC
ACCTGCAGGAGACCAGCCCGCTGAGGGATGCCGTCGTCATCGAGCAGAGCTCCT
CCCTGAATGAGGCCAGCAGTGGCCTCTTCGACGTCTTCCTACGCTTCATGTGCCA
CCACGCCGTGCGCATCAGGGGCAAGTCCTACGTCCAGTGCCAGGGGATCCCGCA
GGGCTCCATCCTCTCCACGCTGCTCTGCAGCCTGTGCTACGGCGACATGGAGAAC
AAGCTGTTTGCGGGGATTCGGCGGGACGGGCTGCTCCTGCGTTTGGTGGATGATT
TCTTGTTGGTGACACCTCACCTCACCCACGCGAAAACCTTCCTCAGCTATGCCCG
GACCTCCATCAGAGCCAGTCTCACCTTCAACCGCGGCTTCAAGGCTGGGAGGAA
CATGCGTCGCAAACTCTTTGGGGTCTTGCGGCTGAAGTGTCACAGCCTGTTTCTG
GATTTGCAGGTGAACAGCCTCCAGACGGTGTGCACCAACATCTACAAGATCCTCC
TGCTGCAGGCGTACAGGTTTCACGCATGTGTGCTGCAGCTCCCATTTCATCAGCA
AGTTTGGAAGAACCCCACATTTTTCCTGCGCGTCATCTCTGACACGGCCTCCCTCT
GCTACTCCATCCTGAAAGCCAAGAACGCAGGGATGTCGCTGGGGGCCAAGGGCG
CCGCCGGCCCTCTGCCCTCCGAGGCCGTGCAGTGGCTGTGCCACCAAGCATTCCT
GCTCAAGCTGACTCGACACCGTGTCACCTACGTGCCACTCCTGGGGTCACTCAGG
ACAGCCCAGACGCAGCTGAGTCGGAAGCTCCCGGGGACGACGCTGACTGCCCTG
GAGGCCGCAGCCAACCCGGCACTGCCCTCAGACTTCAAGACCATCCTGGACTGA
TGGCCACCCGCCCACAGCCAGGCCGAGAGCAGACACCAGCAGCCCTGTCACGCC
GGGCTCTACGTCCCAGGGAGGGAGGGGCGGCCCACACCCAGGCCCGCACCGCTG
GGAGTCTGAGGCCTGAGTGAGTGTTTGGCCGAGGCCTGCATGTCCGGCTGAAGG
CTGAGTGTCCGGCTGAGGCCTGAGCGAGTGTCCAGCCAAGGGCTGAGTGTCCAG
CACACCTGCCGTCTTCACTTCCCCACAGGCTGGCGCTCGGCTCCACCCCAGGGCC
AGCTTTTCCTCACCAGGAGCCCGGCTTCCACTCCCCACATAGGAATAGTCCATCC
CCAGATTCGCCATTGTTCACCCCTCGCCCTGCCCTCCTTTGCCTTCCACCCCCACC
ATCCAGGTGGAGACCCTGAGAAGGACCCTGGGAGCTCTGGGAATTTGGAGTGAC
CAAAGGTGTGCCCTGTACACAGGCGAGGACCCTGCACCTGGATGGGGGTCCCTG
TGGGTCAAATTGGGGGGAGGTGCTGTGGGAGTAAAATACTGAATATATGAGTTT
TTCAGTTTTGAAAAAAA.

Human Telomerase reverse transcriptase isoform 2, NCBI Reference Sequence: NP_001180305.1:

(SEQ ID NO: 2)
MPRAPRCRAVRSLLRSHYREVLPLATFVRRLGPQGWRLVQRGDPAAFRA
LVAQCLVCVPWDARPPPAAPSFRQVSCLKELVARVLQRLCERGAKNVLA
FGFALLDGARGGPPEAFTTSVRSYLPNTVTDALRGSGAWGLLLRRVGDD
VLVHLLARCALFVLVAPSCAYQVCGPPLYQLGAATQARPPPHASGPRRR
LGCERAWNHSVREAGVPLGLPAPGARRRGGSASRSLPLPKRPRRGAAPE
PERTPVGQGSWAHPGRTRGPSDRGFCVVSPARPAEEATSLEGALSGTRH
SHPSVGRQHHAGPPSTSRPPRPWDTPCPPVYAETKHFLYSSGDKEQLRP
SFLLSSLRPSLTGARRLVETIFLGSRPWMPGTPRRLPRLPQRYWQMRPL
FLELLGNHAQCPYGVLLKTHCPLRAAVTPAAGVCAREKPQGSVAAPEEE
DTDPRRLVQLLRQHSSPWQVYGFVRACLRRLVPPGLWGSRHNERRFLRN
TKKFISLGKHAKLSLQELTWKMSVRDCAWLRRSPGVGCVPAAEHRLREE
ILAKFLHWLMSVYVVELLRSFFYVTETTFQKNRLFFYRKSVWSKLQSIG
IRQHLKRVQLRELSEAEVRQHREARPALLTSRLRFIPKPDGLRPIVNMD
YVVGARTFRREKRAERLTSRVKALFSVLNYERARRPGLLGASVLGLDDI
HRAWRTFVLRVRAQDPPPELYFVKVDVTGAYDTIPQDRLTEVIASIIKP
QNTYCVRRYAVVQKAAHGHVRKAFKSHVSTLTDLQPYMRQFVAHLQETS
PLRDAVVIEQSSSLNEASSGLFDVFLRFMCHHAVRIRGKSYVQCQGIPQ
GSILSTLLCSLCYGDMENKLFAGIRRDGLLLRLVDDFLLVTPHLTHAKT
FLSYARTSIRASLTFNRGFKAGRNMRRKLFGVLRLKCHSLFLDLQVNSL
QTVCTNIYKILLLQAYRFHACVLQLPFHQQVWKNPTFFLRVISDTASLC
YSILKAKNAGMSLGAKGAAGPLPSEAVQWLCHQAFLLKLTRHRVTYVPL
LGSLRTAQTQLSRKLPGTTLTALEAAANPALPSDFKTILD.

Homo sapiens telomerase reverse transcriptase (TERT), transcript variant 1, mRNA, NCBI Reference Sequence: NM 198253.2:

(SEQ ID NO: 3)
CAGGCAGCGCTGCGTCCTGCTGCGCACGTGGGAAGCCCTGGCCCCGGCCACCCC
CGCGATGCCGCGCGCTCCCCGCTGCCGAGCCGTGCGCTCCCTGCTGCGCAGCCAC
TACCGCGAGGTGCTGCCGCTGGCCACGTTCGTGCGGCGCCTGGGGCCCCAGGGC
TGGCGGCTGGTGCAGCGCGGGGACCCGGCGGCTTTCCGCGCGCTGGTGGCCCAG
TGCCTGGTGTGCGTGCCCTGGGACGCACGGCCGCCCCCCGCCGCCCCCTCCTTCC
GCCAGGTGTCCTGCCTGAAGGAGCTGGTGGCCCGAGTGCTGCAGAGGCTGTGCG
AGCGCGGCGCGAAGAACGTGCTGGCCTTCGGCTTCGCGCTGCTGGACGGGGCCC
GCGGGGGCCCCCCCGAGGCCTTCACCACCAGCGTGCGCAGCTACCTGCCCAACA
CGGTGACCGACGCACTGCGGGGGAGCGGGGCGTGGGGGCTGCTGCTGCGCCGCG
TGGGCGACGACGTGCTGGTTCACCTGCTGGCACGCTGCGCGCTCTTTGTGCTGGT
GGCTCCCAGCTGCGCCTACCAGGTGTGCGGGCCGCCGCTGTACCAGCTCGGCGCT
GCCACTCAGGCCCGGCCCCCGCCACACGCTAGTGGACCCCGAAGGCGTCTGGGA
TGCGAACGGGCCTGGAACCATAGCGTCAGGGAGGCCGGGGTCCCCCTGGGCCTG
CCAGCCCCGGGTGCGAGGAGGCGCGGGGGCAGTGCCAGCCGAAGTCTGCCGTTG
CCCAAGAGGCCCAGGCGTGGCGCTGCCCCTGAGCCGGAGCGGACGCCCGTTGGG
CAGGGGTCCTGGGCCCACCCGGGCAGGACGCGTGGACCGAGTGACCGTGGTTTC
TGTGTGGTGTCACCTGCCAGACCCGCCGAAGAAGCCACCTCTTTGGAGGGTGCGC
TCTCTGGCACGCGCCACTCCCACCCATCCGTGGGCCGCCAGCACCACGCGGGCCC
CCCATCCACATCGCGGCCACCACGTCCCTGGGACACGCCTTGTCCCCCGGTGTAC
GCCGAGACCAAGCACTTCCTCTACTCCTCAGGCGACAAGGAGCAGCTGCGGCCC
TCCTTCCTACTCAGCTCTCTGAGGCCCAGCCTGACTGGCGCTCGGAGGCTCGTGG
AGACCATCTTTCTGGGTTCCAGGCCCTGGATGCCAGGGACTCCCCGCAGGTTGCC
CCGCCTGCCCCAGCGCTACTGGCAAATGCGGCCCCTGTTTCTGGAGCTGCTTGGG
AACCACGCGCAGTGCCCCTACGGGGTGCTCCTCAAGACGCACTGCCCGCTGCGA
GCTGCGGTCACCCCAGCAGCCGGTGTCTGTGCCCGGGAGAAGCCCCAGGGCTCT
GTGGCGGCCCCCGAGGAGGAGGACACAGACCCCCGTCGCCTGGTGCAGCTGCTC
CGCCAGCACAGCAGCCCCTGGCAGGTGTACGGCTTCGTGCGGGCCTGCCTGCGC
CGGCTGGTGCCCCCAGGCCTCTGGGGCTCCAGGCACAACGAACGCCGCTTCCTCA
GGAACACCAAGAAGTTCATCTCCCTGGGGAAGCATGCCAAGCTCTCGCTGCAGG
AGCTGACGTGGAAGATGAGCGTGCGGGACTGCGCTTGGCTGCGCAGGAGCCCAG
GGGTTGGCTGTGTTCCGGCCGCAGAGCACCGTCTGCGTGAGGAGATCCTGGCCA
AGTTCCTGCACTGGCTGATGAGTGTGTACGTCGTCGAGCTGCTCAGGTCTTTCTTT
TATGTCACGGAGACCACGTTTCAAAAGAACAGGCTCTTTTTCTACCGGAAGAGTG
TCTGGAGCAAGTTGCAAAGCATTGGAATCAGACAGCACTTGAAGAGGGTGCAGC
TGCGGGAGCTGTCGGAAGCAGAGGTCAGGCAGCATCGGGAAGCCAGGCCCGCCC
TGCTGACGTCCAGACTCCGCTTCATCCCCAAGCCTGACGGGCTGCGGCCGATTGT
GAACATGGACTACGTCGTGGGAGCCAGAACGTTCCGCAGAGAAAAGAGGGCCG
AGCGTCTCACCTCGAGGGTGAAGGCACTGTTCAGCGTGCTCAACTACGAGCGGG
CGCGGCGCCCCGGCCTCCTGGGCGCCTCTGTGCTGGGCCTGGACGATATCCACAG
GGCCTGGCGCACCTTCGTGCTGCGTGTGCGGGCCCAGGACCCGCCGCCTGAGCTG
TACTTTGTCAAGGTGGATGTGACGGGCGCGTACGACACCATCCCCCAGGACAGG
CTCACGGAGGTCATCGCCAGCATCATCAAACCCCAGAACACGTACTGCGTGCGT
CGGTATGCCGTGGTCCAGAAGGCCGCCCATGGGCACGTCCGCAAGGCCTTCAAG
AGCCACGTCTCTACCTTGACAGACCTCCAGCCGTACATGCGACAGTTCGTGGCTC
ACCTGCAGGAGACCAGCCCGCTGAGGGATGCCGTCGTCATCGAGCAGAGCTCCT
CCCTGAATGAGGCCAGCAGTGGCCTCTTCGACGTCTTCCTACGCTTCATGTGCCA
CCACGCCGTGCGCATCAGGGGCAAGTCCTACGTCCAGTGCCAGGGGATCCCGCA
GGGCTCCATCCTCTCCACGCTGCTCTGCAGCCTGTGCTACGGCGACATGGAGAAC
AAGCTGTTTGCGGGGATTCGGCGGGACGGGCTGCTCCTGCGTTTGGTGGATGATT
TCTTGTTGGTGACACCTCACCTCACCCACGCGAAAACCTTCCTCAGGACCCTGGT
CCGAGGTGTCCCTGAGTATGGCTGCGTGGTGAACTTGCGGAAGACAGTGGTGAA
CTTCCCTGTAGAAGACGAGGCCCTGGGTGGCACGGCTTTTGTTCAGATGCCGGCC
CACGGCCTATTCCCCTGGTGCGGCCTGCTGCTGGATACCCGGACCCTGGAGGTGC
AGAGCGACTACTCCAGCTATGCCCGGACCTCCATCAGAGCCAGTCTCACCTTCAA
CCGCGGCTTCAAGGCTGGGAGGAACATGCGTCGCAAACTCTTTGGGGTCTTGCGG
CTGAAGTGTCACAGCCTGTTTCTGGATTTGCAGGTGAACAGCCTCCAGACGGTGT
GCACCAACATCTACAAGATCCTCCTGCTGCAGGCGTACAGGTTTCACGCATGTGT
GCTGCAGCTCCCATTTCATCAGCAAGTTTGGAAGAACCCCACATTTTTCCTGCGC
GTCATCTCTGACACGGCCTCCCTCTGCTACTCCATCCTGAAAGCCAAGAACGCAG
GGATGTCGCTGGGGGCCAAGGGCGCCGCCGGCCCTCTGCCCTCCGAGGCCGTGC
AGTGGCTGTGCCACCAAGCATTCCTGCTCAAGCTGACTCGACACCGTGTCACCTA
CGTGCCACTCCTGGGGTCACTCAGGACAGCCCAGACGCAGCTGAGTCGGAAGCT
CCCGGGGACGACGCTGACTGCCCTGGAGGCCGCAGCCAACCCGGCACTGCCCTC
AGACTTCAAGACCATCCTGGACTGATGGCCACCCGCCCACAGCCAGGCCGAGAG
CAGACACCAGCAGCCCTGTCACGCCGGGCTCTACGTCCCAGGGAGGGAGGGGCG
GCCCACACCCAGGCCCGCACCGCTGGGAGTCTGAGGCCTGAGTGAGTGTTTGGC
CGAGGCCTGCATGTCCGGCTGAAGGCTGAGTGTCCGGCTGAGGCCTGAGCGAGT
GTCCAGCCAAGGGCTGAGTGTCCAGCACACCTGCCGTCTTCACTTCCCCACAGGC
TGGCGCTCGGCTCCACCCCAGGGCCAGCTTTTCCTCACCAGGAGCCCGGCTTCCA
CTCCCCACATAGGAATAGTCCATCCCCAGATTCGCCATTGTTCACCCCTCGCCCT
GCCCTCCTTTGCCTTCCACCCCCACCATCCAGGTGGAGACCCTGAGAAGGACCCT
GGGAGCTCTGGGAATTTGGAGTGACCAAAGGTGTGCCCTGTACACAGGCGAGGA
CCCTGCACCTGGATGGGGGTCCCTGTGGGTCAAATTGGGGGGAGGTGCTGTGGG
AGTAAAATACTGAATATATGAGTTTTTCAGTTTTGAAAAAAA.

Human telomerase reverse transcriptase isoform 1, NCBI reference sequence: NP_937983.2:

(SEQ ID NO: 4)
MPRAPRCRAVRSLLRSHYREVLPLATFVRRLGPQGWRLVQRGDPAAFRA
LVAQCLVCVPWDARPPPAAPSFRQVSCLKELVARVLQRLCERGAKNVLA
FGFALLDGARGGPPEAFTTSVRSYLPNTVTDALRGSGAWGLLLRRVGDD
VLVHLLARCALFVLVAPSCAYQVCGPPLYQLGAATQARPPPHASGPRRR
LGCERAWNHSVREAGVPLGLPAPGARRRGGSASRSLPLPKRPRRGAAPE
PERTPVGQGSWAHPGRTRGPSDRGFCVVSPARPAEEATSLEGALSGTRH
SHPSVGRQHHAGPPSTSRPPRPWDTPCPPVYAETKHFLYSSGDKEQLRP
SFLLSSLRPSLTGARRLVETIFLGSRPWMPGTPRRLPRLPQRYWQMRPL
FLELLGNHAQCPYGVLLKTHCPLRAAVTPAAGVCAREKPQGSVAAPEEE
DTDPRRLVQLLRQHSSPWQVYGFVRACLRRLVPPGLWGSRHNERRFLRN
TKKFISLGKHAKLSLQELTWKMSVRDCAWLRRSPGVGCVPAAEHRLREE
ILAKFLHWLMSVYVVELLRSFFYVTETTFQKNRLFFYRKSVWSKLQSIG
IRQHLKRVQLRELSEAEVRQHREARPALLTSRLRFIPKPDGLRPIVNMD
YVVGARTFRREKRAERLTSRVKALFSVLNYERARRPGLLGASVLGLDDI
HRAWRTFVLRVRAQDPPPELYFVKVDVTGAYDTIPQDRLTEVIASIIKP
QNTYCVRRYAVVQKAAHGHVRKAFKSHVSTLTDLQPYMRQFVAHLQETS
PLRDAVVIEQSSSLNEASSGLFDVFLRFMCHHAVRIRGKSYVQCQGIPQ
GSILSTLLCSLCYGDMENKLFAGIRRDGLLLRLVDDFLLVTPHLTHAKT
FLRTLVRGVPEYGCVVNLRKTVVNFPVEDEALGGTAFVQMPAHGLFPWC
GLLLDTRTLEVQSDYSSYARTSIRASLTFNRGFKAGRNMRRKLFGVLRL
KCHSLFLDLQVNSLQTVCTNIYKILLLQAYRFHACVLQLPFHQQVWKNP
TFFLRVISDTASLCYSILKAKNAGMSLGAKGAAGPLPSEAVQWLCHQAF
LLKLTRHRVTYVPLLGSLRTAQTQLSRKLPGTTLTALEAAANPALPSDF
KTILD.

Mus musculus telomerase reverse transcriptase (Tert), transcript variant 2, mRNA, NCBI Reference Sequence: NM_001362387.1:

(SEQ ID NO: 5)
GTTCCCAGCCTCATCTTTTTCGTCGTGGACTCTCAGTGGCCTGGGTCCTGGCTGTT
TTCTAAGCACACCCTTGCATCTTGGTTCCCGCACGTGGGAGGCCCATCCCGGCCT
TGAGCACAATGACCCGCGCTCCTCGTTGCCCCGCGGTGCGCTCTCTGCTGCGCAG
CCGATACCGGGAGGTGTGGCCGCTGGCAACCTTTGTGCGGCGCCTGGGGCCCGA
GGGCAGGCGGCTTGTGCAACCCGGGGACCCGAAGATCTACCGCACTTTGGTTGC
CCAATGCCTAGTGTGCATGCACTGGGGCTCACAGCCTCCACCTGCCGACCTTTCC
TTCCACCAGGTGTCATCCCTGAAAGAGCTGGTGGCCAGGGTTGTGCAGAGACTCT
GCGAGCGCAACGAGAGAAACGTGCTGGCTTTTGGCTTTGAGCTGCTTAACGAGG
CCAGAGGCGGGCCTCCCATGGCCTTCACTAGTAGCGTGCGTAGCTACTTGCCCAA
CACTGTTATTGAGACCCTGCGTGTCAGTGGTGCATGGATGCTACTGTTGAGCCGA
GTGGGCGACGACCTGCTGGTCTACCTGCTGGCACACTGTGCTCTTTATCTTCTGGT
GCCCCCCAGCTGTGCCTACCAGGGGAGATGGCCAAGAGCGTCTAAACCCCTCAT
TCCTACTCAGCAACCTCCAGCCTAACTTGACTGGGGCCAGGAGACTGGTGGAGAT
CATCTTTCTGGGCTCAAGGCCTAGGACATCAGGACCACTCTGCAGGACACACCGT
CTATCGCGTCGATACTGGCAGATGCGGCCCCTGTTCCAACAGCTGCTGGTGAACC
ATGCAGAGTGCCAATATGTCAGACTCCTCAGGTCACATTGCAGGTTTCGAACAGC
AAACCAACAGGTGACAGATGCCTTGAACACCAGCCCACCGCACCTCATGGATTT
GCTCCGCCTGCACAGCAGTCCCTGGCAGGTATATGGTTTTCTTCGGGCCTGTCTCT
GCAAGGTGGTGTCTGCTAGTCTCTGGGGTACCAGGCACAATGAGCGCCGCTTCTT
TAAGAACTTAAAGAAGTTCATCTCGTTGGGGAAATACGGCAAGCTATCACTGCA
GGAACTGATGTGGAAGATGAAAGTAGAGGATTGCCACTGGCTCCGCAGCAGCCC
GGGGAAGGACCGTGTCCCCGCTGCAGAGCACCGTCTGAGGGAGAGGATCCTGGC
TACGTTCCTGTTCTGGCTGATGGACACATACGTGGTACAGCTGCTTAGGTCATTCT
TTTACATCACAGAGAGCACATTCCAGAAGAACAGGCTCTTCTTCTACCGTAAGAG
TGTGTGGAGCAAGCTGCAGAGCATTGGAGTCAGGCAACACCTTGAGAGAGTGCG
GCTACGGGAGCTGTCACAAGAGGAGGTCAGGCATCACCAGGACACCTGGCTAGC
CATGCCCATCTGCAGACTGCGCTTCATCCCCAAGCCCAACGGCCTGCGGCCCATT
GTGAACATGAGTTATAGCATGGGTACCAGAGCTTTGGGCAGAAGGAAGCAGGCC
CAGCATTTCACCCAGCGTCTCAAGACTCTCTTCAGCATGCTCAACTATGAGCGGA
CAAAACATCCTCACCTTATGGGGTCTTCTGTACTGGGTATGAATGACATCTACAG
GACCTGGCGGGCCTTTGTGCTGCGTGTGCGTGCTCTGGACCAGACACCCAGGATG
TACTTTGTTAAGGCAGATGTGACCGGGGCCTATGATGCCATCCCCCAGGGTAAGC
TGGTGGAGGTTGTTGCCAATATGATCAGGCACTCGGAGAGCACGTACTGTATCCG
CCAGTATGCAGTGGTCCGGAGAGATAGCCAAGGCCAAGTCCACAAGTCCTTTAG
GAGACAGGTCACCACCCTCTCTGACCTCCAGCCATACATGGGCCAGTTCCTTAAG
CATCTGCAGGATTCAGATGCCAGTGCACTGAGGAACTCCGTTGTCATCGAGCAGA
GCATCTCTATGAATGAGAGCAGCAGCAGCCTGTTTGACTTCTTCCTGCACTTCCT
GCGTCACAGTGTCGTAAAGATTGGTGACAGGTGCTATACGCAGTGCCAGGGCAT
CCCCCAGGGCTCCAGCCTATCCACCCTGCTCTGCAGTCTGTGTTTCGGAGACATG
GAGAACAAGCTGTTTGCTGAGGTGCAGCGGGATGGGTTGCTTTTACGTTTTGTTG
ATGACTTTCTGTTGGTGACGCCTCACTTGGACCAAGCAAAAACCTTCCTCAGCAC
CCTGGTCCATGGCGTTCCTGAGTATGGGTGCATGATAAACTTGCAGAAGACAGTG
GTGAACTTCCCTGTGGAGCCTGGTACCCTGGGTGGTGCAGCTCCATACCAGCTGC
CTGCTCACTGCCTGTTTCCCTGGTGTGGCTTGCTGCTGGACACTCAGACTTTGGAG
GTGTTCTGTGACTACTCAGGTTATGCCCAGACCTCAATTAAGACGAGCCTCACCT
TCCAGAGTGTCTTCAAAGCTGGGAAGACCATGCGGAACAAGCTCCTGTCGGTCTT
GCGGTTGAAGTGTCACGGTCTATTTCTAGACTTGCAGGTGAACAGCCTCCAGACA
GTCTGCATCAATATATACAAGATCTTCCTGCTTCAGGCCTACAGGTTCCATGCAT
GTGTGATTCAGCTTCCCTTTGACCAGCGTGTTAGGAAGAACCTCACATTCTTTCTG
GGCATCATCTCCAGCCAAGCATCCTGCTGCTATGCTATCCTGAAGGTCAAGAATC
CAGGAATGACACTAAAGGCCTCTGGCTCCTTTCCTCCTGAAGCCGCACATTGGCT
CTGCTACCAGGCCTTCCTGCTCAAGCTGGCTGCTCATTCTGTCATCTACAAATGTC
TCCTGGGACCTCTGAGGACAGCCCAAAAACTGCTGTGCCGGAAGCTCCCAGAGG
CGACAATGACCATCCTTAAAGCTGCAGCTGACCCAGCCCTAAGCACAGACTTTCA
GACCATTTTGGACTAACCCTGTCTCCTTCCGCTAGATGAACATGGGCATTGTAGC
CTCAGCACTCCTGGATCCACGTCACAAGAGGGACTGGTCAGTTGTGAGGCTAGGT
CATCCTCCAAACCTCTGTGTCATGGGTGGTATGGGAGATTGTCCCAGTGCCTTGT
TTCCTGTAACAGGCTTGATTTCTTTCCTGATGCCCTCAGGGAGGCAGATCCTATCC
CTTTTAGTGGCAGGGATCCACTAGCACCAGCACATGAGGAGTGCACCCAGTGCA
CATGGGCACTGGGACAGTGGACAGGTGTGAGATTCCTGGGCCCTGGAGTCTTTTC
ACACCTAACCATGGAGCCTGTCCCAGTACATCAGAGTGCCTCGGAGATGAAAAA
GGACATCGAGCCAGTGACCTAAATTACAGCCTGAATATACTCTGAATTCATGTGA
CTGCCTTAGCTACTTCTCTACTGCTGTGTAGTAAAACACCAAGCCAACTTATAAA
AGCAGGATTTTCCTACTGGAGCAGCAGCTGAGAGTTTACATCTTGATCCATAAGC
ACAAAAGCACAAGACAGAGAGAGAGAGAGAGAGAGAGAGAGAGAGAGAGAGA
GAGAGAGAGAGAGAGAGAGAGAGTCAGTCAGTCAGTCTAACAAATAACTAAGA
AAGGTGAAGGGTGATGAAGTCCACAGGGATCACGCTAGGGATGTTCCATGCCTT
CTCTGAAGCTAAGATTCCTTGGCAGCGTTTGACAGTAACCATAGTGGGTACCTAC
TGAGATCACTATAAAGATAAAATAGGGGGAAGCGTATTTGTACTGAACTGGAAA
AACATACAAATAAAGAGTAAATCATGGAAAAAAAAAAAAAAAAAAA.

Mus musculus telomerase reverse transcriptase isoform 2, NCBI Reference Sequence: NP_001349316.1:

(SEQ ID NO: 6)
MRPLFQQLLVNHAECQYVRLLRSHCRFRTANQQVTDALNTSPPHLMDLL
RLHSSPWQVYGFLRACLCKVVSASLWGTRHNERRFFKNLKKFISLGKYG
KLSLQELMWKMKVEDCHWLRSSPGKDRVPAAEHRLRERILATFLFWLMD
TYVVQLLRSFFYITESTFQKNRLFFYRKSVWSKLQSIGVRQHLERVRLR
ELSQEEVRHHQDTWLAMPICRLRFIPKPNGLRPIVNMSYSMGTRALGRR
KQAQHFTQRLKTLFSMLNYERTKHPHLMGSSVLGMNDIYRTWRAFVLRV
RALDQTPRMYFVKADVTGAYDAIPQGKLVEVVANMIRHSESTYCIRQYA
VVRRDSQGQVHKSFRRQVTTLSDLQPYMGQFLKHLQDSDASALRNSVVI
EQSISMNESSSSLFDFFLHFLRHSVVKIGDRCYTQCQGIPQGSSLSTLL
CSLCFGDMENKLFAEVQRDGLLLRFVDDFLLVTPHLDQAKTFLSTLVHG
VPEYGCMINLQKTVVNFPVEPGTLGGAAPYQLPAHCLFPWCGLLLDTQT
LEVFCDYSGYAQTSIKTSLTFQSVFKAGKTMRNKLLSVLRLKCHGLFLD
LQVNSLQTVCINIYKIFLLQAYRFHACVIQLPFDQRVRKNLTFFLGIIS
SQASCCYAILKVKNPGMTLKASGSFPPEAAHWLCYQAFLLKLAAHSVIY
KCLLGPLRTAQKLLCRKLPEATMTILKAAADPALSTDFQTILD.

Mus musculus telomerase reverse transcriptase (Tert), transcript variant 3, mRNA, NCBI Reference Sequence: NM_001362388.1:

(SEQ ID NO: 7)
GTTCCCAGCCTCATCTTTTTCGTCGTGGACTCTCAGTGGCCTGGGTCCTGGCTGTT
TTCTAAGCACACCCTTGCATCTTGGTTCCCGCACGTGGGAGGCCCATCCCGGCCT
TGAGCACAATGACCCGCGCTCCTCGTTGCCCCGCGGTGCGCTCTCTGCTGCGCAG
CCGATACCGGGAGGTGTGGCCGCTGGCAACCTTTGTGCGGCGCCTGGGGCCCGA
GGGCAGGCGGCTTGTGCAACCCGGGGACCCGAAGATCTACCGCACTTTGGTTGC
CCAATGCCTAGTGTGCATGCACTGGGGCTCACAGCCTCCACCTGCCGACCTTTCC
TTCCACCAGGTGTCATCCCTGAAAGAGCTGGTGGCCAGGGTTGTGCAGAGACTCT
GCGAGCGCAACGAGAGAAACGTGCTGGCTTTTGGCTTTGAGCTGCTTAACGAGG
CCAGAGGCGGGCCTCCCATGGCCTTCACTAGTAGCGTGCGTAGCTACTTGCCCAA
CACTGTTATTGAGACCCTGCGTGTCAGTGGTGCATGGATGCTACTGTTGAGCCGA
GTGGGCGACGACCTGCTGGTCTACCTGCTGGCACACTGTGCTCTTTATCTTCTGGT
GCCCCCCAGCTGTGCCTACCAGGGGAGATGGCCAAGAGCGTCTAAACCCCTCAT
TCCTACTCAGCAACCTCCAGCCTAACTTGACTGGGGCCAGGAGACTGGTGGAGAT
CATCTTTCTGGGCTCAAGGCCTAGGACATCAGGACCACTCTGCAGGACACACCGT
CTATCGCGTCGATACTGGCAGATGCGGCCCCTGTTCCAACAGCTGCTGGTGAACC
ATGCAGAGTGCCAATATGTCAGACTCCTCAGGTCACATTGCAGGTTTCGAACAGC
AAACCAACAGGTGACAGATGCCTTGAACACCAGCCCACCGCACCTCATGGATTT
GCTCCGCCTGCACAGCAGTCCCTGGCAGGGAAGGACCGTGTCCCCGCTGCAGAG
CACCGTCTGAGGGAGAGGATCCTGGCTACGTTCCTGTTCTGGCTGATGGACACAT
ACGTGGTACAGCTGCTTAGGTCATTCTTTTACATCACAGAGAGCACATTCCAGAA
GAACAGGCTCTTCTTCTACCGTAAGAGTGTGTGGAGCAAGCTGCAGAGCATTGG
AGTCAGGCAACACCTTGAGAGAGTGCGGCTACGGGAGCTGTCACAAGAGGAGGT
CAGGCATCACCAGGACACCTGGCTAGCCATGCCCATCTGCAGACTGCGCTTCATC
CCCAAGCCCAACGGCCTGCGGCCCATTGTGAACATGAGTTATAGCATGGGTACC
AGAGCTTTGGGCAGAAGGAAGCAGGCCCAGCATTTCACCCAGCGTCTCAAGACT
CTCTTCAGCATGCTCAACTATGAGCGGACAAAACATCCTCACCTTATGGGGTCTT
CTGTACTGGGTATGAATGACATCTACAGGACCTGGCGGGCCTTTGTGCTGCGTGT
GCGTGCTCTGGACCAGACACCCAGGATGTACTTTGTTAAGGCAGATGTGACCGG
GGCCTATGATGCCATCCCCCAGGGTAAGCTGGTGGAGGTTGTTGCCAATATGATC
AGGCACTCGGAGAGCACGTACTGTATCCGCCAGTATGCAGTGGTCCGGAGAGAT
AGCCAAGGCCAAGTCCACAAGTCCTTTAGGAGACAGGTCACCACCCTCTCTGAC
CTCCAGCCATACATGGGCCAGTTCCTTAAGCATCTGCAGGATTCAGATGCCAGTG
CACTGAGGAACTCCGTTGTCATCGAGCAGAGCATCTCTATGAATGAGAGCAGCA
GCAGCCTGTTTGACTTCTTCCTGCACTTCCTGCGTCACAGTGTCGTAAAGATTGGT
GACAGGTGCTATACGCAGTGCCAGGGCATCCCCCAGGGCTCCAGCCTATCCACC
CTGCTCTGCAGTCTGTGTTTCGGAGACATGGAGAACAAGCTGTTTGCTGAGGTGC
AGCGGGATGGGTTGCTTTTACGTTTTGTTGATGACTTTCTGTTGGTGACGCCTCAC
TTGGACCAAGCAAAAACCTTCCTCAGCACCCTGGTCCATGGCGTTCCTGAGTATG
GGTGCATGATAAACTTGCAGAAGACAGTGGTGAACTTCCCTGTGGAGCCTGGTA
CCCTGGGTGGTGCAGCTCCATACCAGCTGCCTGCTCACTGCCTGTTTCCCTGGTGT
GGCTTGCTGCTGGACACTCAGACTTTGGAGGTGTTCTGTGACTACTCAGGTTATG
CCCAGACCTCAATTAAGACGAGCCTCACCTTCCAGAGTGTCTTCAAAGCTGGGAA
GACCATGCGGAACAAGCTCCTGTCGGTCTTGCGGTTGAAGTGTCACGGTCTATTT
CTAGACTTGCAGGTGAACAGCCTCCAGACAGTCTGCATCAATATATACAAGATCT
TCCTGCTTCAGGCCTACAGGTTCCATGCATGTGTGATTCAGCTTCCCTTTGACCAG
CGTGTTAGGAAGAACCTCACATTCTTTCTGGGCATCATCTCCAGCCAAGCATCCT
GCTGCTATGCTATCCTGAAGGTCAAGAATCCAGGAATGACACTAAAGGCCTCTG
GCTCCTTTCCTCCTGAAGCCGCACATTGGCTCTGCTACCAGGCCTTCCTGCTCAAG
CTGGCTGCTCATTCTGTCATCTACAAATGTCTCCTGGGACCTCTGAGGACAGCCC
AAAAACTGCTGTGCCGGAAGCTCCCAGAGGCGACAATGACCATCCTTAAAGCTG
CAGCTGACCCAGCCCTAAGCACAGACTTTCAGACCATTTTGGACTAACCCTGTCT
CCTTCCGCTAGATGAACATGGGCATTGTAGCCTCAGCACTCCTGGATCCACGTCA
CAAGAGGGACTGGTCAGTTGTGAGGCTAGGTCATCCTCCAAACCTCTGTGTCATG
GGTGGTATGGGAGATTGTCCCAGTGCCTTGTTTCCTGTAACAGGCTTGATTTCTTT
CCTGATGCCCTCAGGGAGGCAGATCCTATCCCTTTTAGTGGCAGGGATCCACTAG
CACCAGCACATGAGGAGTGCACCCAGTGCACATGGGCACTGGGACAGTGGACAG
GTGTGAGATTCCTGGGCCCTGGAGTCTTTTCACACCTAACCATGGAGCCTGTCCC
AGTACATCAGAGTGCCTCGGAGATGAAAAAGGACATCGAGCCAGTGACCTAAAT
TACAGCCTGAATATACTCTGAATTCATGTGACTGCCTTAGCTACTTCTCTACTGCT
GTGTAGTAAAACACCAAGCCAACTTATAAAAGCAGGATTTTCCTACTGGAGCAG
CAGCTGAGAGTTTACATCTTGATCCATAAGCACAAAAGCACAAGACAGAGAGAG
AGAGAGAGAGAGAGAGAGAGAGAGAGAGAGAGAGAGAGAGAGAGAGAGAGAG
TCAGTCAGTCAGTCTAACAAATAACTAAGAAAGGTGAAGGGTGATGAAGTCCAC
AGGGATCACGCTAGGGATGTTCCATGCCTTCTCTGAAGCTAAGATTCCTTGGCAG
CGTTTGACAGTAACCATAGTGGGTACCTACTGAGATCACTATAAAGATAAAATA
GGGGGAAGCGTATTTGTACTGAACTGGAAAAACATACAAATAAAGAGTAAATCA
TGGAAAAAAAAAAAAAAAAAAA.

Mus musculus telomerase reverse transcriptase isoform 3, NCBI Reference Sequence: NP_001349317.1:

(SEQ ID NO: 8)
MDTYVVQLLRSFFYITESTFQKNRLFFYRKSVWSKLQSIGVRQHLERVR
LRELSQEEVRHHQDTWLAMPICRLRFIPKPNGLRPIVNMSYSMGTRALG
RRKQAQHFTQRLKTLFSMLNYERTKHPHLMGSSVLGMNDIYRTWRAFVL
RVRALDQTPRMYFVKADVTGAYDAIPQGKLVEVVANMIRHSESTYCIRQ
YAVVRRDSQGQVHKSFRRQVTTLSDLQPYMGQFLKHLQDSDASALRNSV
VIEQSISMNESSSSLFDFFLHFLRHSVVKIGDRCYTQCQGIPQGSSLST
LLCSLCFGDMENKLFAEVQRDGLLLRFVDDFLLVTPHLDQAKTFLSTLV
HGVPEYGCMINLQKTVVNFPVEPGTLGGAAPYQLPAHCLFPWCGLLLDT
QTLEVFCDYSGYAQTSIKTSLTFQSVFKAGKTMRNKLLSVLRLKCHGLF
LDLQVNSLQTVCINIYKIFLLQAYRFHACVIQLPFDQRVRKNLTFFLGI
ISSQASCCYAILKVKNPGMTLKASGSFPPEAAHWLCYQAFLLKLAAHSV
IYKCLLGPLRTAQKLLCRKLPEATMTILKAAADPALSTDFQTILD.

Mus musculus telomerase reverse transcriptase (Tert), transcript variant 1, mRNA, NCBI Reference Sequence: NM_009354.2:

(SEQ ID NO: 9)
GTTCCCAGCCTCATCTTTTTCGTCGTGGACTCTCAGTGGCCTGGGTCCTGGCTGTT
TTCTAAGCACACCCTTGCATCTTGGTTCCCGCACGTGGGAGGCCCATCCCGGCCT
TGAGCACAATGACCCGCGCTCCTCGTTGCCCCGCGGTGCGCTCTCTGCTGCGCAG
CCGATACCGGGAGGTGTGGCCGCTGGCAACCTTTGTGCGGCGCCTGGGGCCCGA
GGGCAGGCGGCTTGTGCAACCCGGGGACCCGAAGATCTACCGCACTTTGGTTGC
CCAATGCCTAGTGTGCATGCACTGGGGCTCACAGCCTCCACCTGCCGACCTTTCC
TTCCACCAGGTGTCATCCCTGAAAGAGCTGGTGGCCAGGGTTGTGCAGAGACTCT
GCGAGCGCAACGAGAGAAACGTGCTGGCTTTTGGCTTTGAGCTGCTTAACGAGG
CCAGAGGCGGGCCTCCCATGGCCTTCACTAGTAGCGTGCGTAGCTACTTGCCCAA
CACTGTTATTGAGACCCTGCGTGTCAGTGGTGCATGGATGCTACTGTTGAGCCGA
GTGGGCGACGACCTGCTGGTCTACCTGCTGGCACACTGTGCTCTTTATCTTCTGGT
GCCCCCCAGCTGTGCCTACCAGGTGTGTGGGTCTCCCCTGTACCAAATTTGTGCC
ACCACGGATATCTGGCCCTCTGTGTCCGCTAGTTACAGGCCCACCCGACCCGTGG
GCAGGAATTTCACTAACCTTAGGTTCTTACAACAGATCAAGAGCAGTAGTCGCCA
GGAAGCACCGAAACCCCTGGCCTTGCCATCTCGAGGTACAAAGAGGCATCTGAG
TCTCACCAGTACAAGTGTGCCTTCAGCTAAGAAGGCCAGATGCTATCCTGTCCCG
AGAGTGGAGGAGGGACCCCACAGGCAGGTGCTACCAACCCCATCAGGCAAATCA
TGGGTGCCAAGTCCTGCTCGGTCCCCCGAGGTGCCTACTGCAGAGAAAGATTTGT
CTTCTAAAGGAAAGGTGTCTGACCTGAGTCTCTCTGGGTCGGTGTGCTGTAAACA
CAAGCCCAGCTCCACATCTCTGCTGTCACCACCCCGCCAAAATGCCTTTCAGCTC
AGGCCATTTATTGAGACCAGACATTTCCTTTACTCCAGGGGAGATGGCCAAGAGC
GTCTAAACCCCTCATTCCTACTCAGCAACCTCCAGCCTAACTTGACTGGGGCCAG
GAGACTGGTGGAGATCATCTTTCTGGGCTCAAGGCCTAGGACATCAGGACCACTC
TGCAGGACACACCGTCTATCGCGTCGATACTGGCAGATGCGGCCCCTGTTCCAAC
AGCTGCTGGTGAACCATGCAGAGTGCCAATATGTCAGACTCCTCAGGTCACATTG
CAGGTTTCGAACAGCAAACCAACAGGTGACAGATGCCTTGAACACCAGCCCACC
GCACCTCATGGATTTGCTCCGCCTGCACAGCAGTCCCTGGCAGGTATATGGTTTT
CTTCGGGCCTGTCTCTGCAAGGTGGTGTCTGCTAGTCTCTGGGGTACCAGGCACA
ATGAGCGCCGCTTCTTTAAGAACTTAAAGAAGTTCATCTCGTTGGGGAAATACGG
CAAGCTATCACTGCAGGAACTGATGTGGAAGATGAAAGTAGAGGATTGCCACTG
GCTCCGCAGCAGCCCGGGGAAGGACCGTGTCCCCGCTGCAGAGCACCGTCTGAG
GGAGAGGATCCTGGCTACGTTCCTGTTCTGGCTGATGGACACATACGTGGTACAG
CTGCTTAGGTCATTCTTTTACATCACAGAGAGCACATTCCAGAAGAACAGGCTCT
TCTTCTACCGTAAGAGTGTGTGGAGCAAGCTGCAGAGCATTGGAGTCAGGCAAC
ACCTTGAGAGAGTGCGGCTACGGGAGCTGTCACAAGAGGAGGTCAGGCATCACC
AGGACACCTGGCTAGCCATGCCCATCTGCAGACTGCGCTTCATCCCCAAGCCCAA
CGGCCTGCGGCCCATTGTGAACATGAGTTATAGCATGGGTACCAGAGCTTTGGGC
AGAAGGAAGCAGGCCCAGCATTTCACCCAGCGTCTCAAGACTCTCTTCAGCATG
CTCAACTATGAGCGGACAAAACATCCTCACCTTATGGGGTCTTCTGTACTGGGTA
TGAATGACATCTACAGGACCTGGCGGGCCTTTGTGCTGCGTGTGCGTGCTCTGGA
CCAGACACCCAGGATGTACTTTGTTAAGGCAGATGTGACCGGGGCCTATGATGCC
ATCCCCCAGGGTAAGCTGGTGGAGGTTGTTGCCAATATGATCAGGCACTCGGAG
AGCACGTACTGTATCCGCCAGTATGCAGTGGTCCGGAGAGATAGCCAAGGCCAA
GTCCACAAGTCCTTTAGGAGACAGGTCACCACCCTCTCTGACCTCCAGCCATACA
TGGGCCAGTTCCTTAAGCATCTGCAGGATTCAGATGCCAGTGCACTGAGGAACTC
CGTTGTCATCGAGCAGAGCATCTCTATGAATGAGAGCAGCAGCAGCCTGTTTGAC
TTCTTCCTGCACTTCCTGCGTCACAGTGTCGTAAAGATTGGTGACAGGTGCTATA
CGCAGTGCCAGGGCATCCCCCAGGGCTCCAGCCTATCCACCCTGCTCTGCAGTCT
GTGTTTCGGAGACATGGAGAACAAGCTGTTTGCTGAGGTGCAGCGGGATGGGTT
GCTTTTACGTTTTGTTGATGACTTTCTGTTGGTGACGCCTCACTTGGACCAAGCAA
AAACCTTCCTCAGCACCCTGGTCCATGGCGTTCCTGAGTATGGGTGCATGATAAA
CTTGCAGAAGACAGTGGTGAACTTCCCTGTGGAGCCTGGTACCCTGGGTGGTGCA
GCTCCATACCAGCTGCCTGCTCACTGCCTGTTTCCCTGGTGTGGCTTGCTGCTGGA
CACTCAGACTTTGGAGGTGTTCTGTGACTACTCAGGTTATGCCCAGACCTCAATT
AAGACGAGCCTCACCTTCCAGAGTGTCTTCAAAGCTGGGAAGACCATGCGGAAC
AAGCTCCTGTCGGTCTTGCGGTTGAAGTGTCACGGTCTATTTCTAGACTTGCAGG
TGAACAGCCTCCAGACAGTCTGCATCAATATATACAAGATCTTCCTGCTTCAGGC
CTACAGGTTCCATGCATGTGTGATTCAGCTTCCCTTTGACCAGCGTGTTAGGAAG
AACCTCACATTCTTTCTGGGCATCATCTCCAGCCAAGCATCCTGCTGCTATGCTAT
CCTGAAGGTCAAGAATCCAGGAATGACACTAAAGGCCTCTGGCTCCTTTCCTCCT
GAAGCCGCACATTGGCTCTGCTACCAGGCCTTCCTGCTCAAGCTGGCTGCTCATT
CTGTCATCTACAAATGTCTCCTGGGACCTCTGAGGACAGCCCAAAAACTGCTGTG
CCGGAAGCTCCCAGAGGCGACAATGACCATCCTTAAAGCTGCAGCTGACCCAGC
CCTAAGCACAGACTTTCAGACCATTTTGGACTAACCCTGTCTCCTTCCGCTAGAT
GAACATGGGCATTGTAGCCTCAGCACTCCTGGATCCACGTCACAAGAGGGACTG
GTCAGTTGTGAGGCTAGGTCATCCTCCAAACCTCTGTGTCATGGGTGGTATGGGA
GATTGTCCCAGTGCCTTGTTTCCTGTAACAGGCTTGATTTCTTTCCTGATGCCCTC
AGGGAGGCAGATCCTATCCCTTTTAGTGGCAGGGATCCACTAGCACCAGCACAT
GAGGAGTGCACCCAGTGCACATGGGCACTGGGACAGTGGACAGGTGTGAGATTC
CTGGGCCCTGGAGTCTTTTCACACCTAACCATGGAGCCTGTCCCAGTACATCAGA
GTGCCTCGGAGATGAAAAAGGACATCGAGCCAGTGACCTAAATTACAGCCTGAA
TATACTCTGAATTCATGTGACTGCCTTAGCTACTTCTCTACTGCTGTGTAGTAAAA
CACCAAGCCAACTTATAAAAGCAGGATTTTCCTACTGGAGCAGCAGCTGAGAGT
TTACATCTTGATCCATAAGCACAAAAGCACAAGACAGAGAGAGAGAGAGAGAG
AGAGAGAGAGAGAGAGAGAGAGAGAGAGAGAGAGAGAGAGAGTCAGTCAGTC
AGTCTAACAAATAACTAAGAAAGGTGAAGGGTGATGAAGTCCACAGGGATCACG
CTAGGGATGTTCCATGCCTTCTCTGAAGCTAAGATTCCTTGGCAGCGTTTGACAG
TAACCATAGTGGGTACCTACTGAGATCACTATAAAGATAAAATAGGGGGAAGCG
TATTTGTACTGAACTGGAAAAACATACAAATAAAGAGTAAATCATGGAAAAAAA
AAAAAAAAAAAA.

Mus musculus telomerase reverse transcriptase isoform 1, NCBI Reference Sequence: NP_033380.1:

(SEQ ID NO: 10)
MTRAPRCPAVRSLLRSRYREVWPLATFVRRLGPEGRRLVQPGDPKIYRT
LVAQCLVCMHWGSQPPPADLSFHQVSSLKELVARVVQRLCERNERNVLA
FGFELLNEARGGPPMAFTSSVRSYLPNTVIETLRVSGAWMLLLSRVGDD
LLVYLLAHCALYLLVPPSCAYQVCGSPLYQICATTDIWPSVSASYRPTR
PVGRNFTNLRFLQQIKSSSRQEAPKPLALPSRGTKRHLSLTSTSVPSAK
KARCYPVPRVEEGPHRQVLPTPSGKSWVPSPARSPEVPTAEKDLSSKGK
VSDLSLSGSVCCKHKPSSTSLLSPPRQNAFQLRPFIETRHFLYSRGDGQ
ERLNPSFLLSNLQPNLTGARRLVEIIFLGSRPRTSGPLCRTHRLSRRYW
QMRPLFQQLLVNHAECQYVRLLRSHCRFRTANQQVTDALNTSPPHLMDL
LRLHSSPWQVYGFLRACLCKVVSASLWGTRHNERRFFKNLKKFISLGKY
GKLSLQELMWKMKVEDCHWLRSSPGKDRVPAAEHRLRERILATFLFWLM
DTYVVQLLRSFFYITESTFQKNRLFFYRKSVWSKLQSIGVRQHLERVRL
RELSQEEVRHHQDTWLAMPICRLRFIPKPNGLRPIVNMSYSMGTRALGR
RKQAQHFTQRLKTLFSMLNYERTKHPHLMGSSVLGMNDIYRTWRAFVLR
VRALDQTPRMYFVKADVTGAYDAIPQGKLVEVVANMIRHSESTYCIRQY
AVVRRDSQGQVHKSFRRQVTTLSDLQPYMGQFLKHLQDSDASALRNSVV
IEQSISMNESSSSLFDFFLHFLRHSVVKIGDRCYTQCQGIPQGSSLSTL
LCSLCFGDMENKLFAEVQRDGLLLRFVDDFLLVTPHLDQAKTFLSTLVH
GVPEYGCMINLQKTVVNFPVEPGTLGGAAPYQLPAHCLFPWCGLLLDTQ
TLEVFCDYSGYAQTSIKTSLTFQSVFKAGKTMRNKLLSVLRLKCHGLFL
DLQVNSLQTVCINIYKIFLLQAYRFHACVIQLPFDQRVRKNLTFFLGII
SSQASCCYAILKVKNPGMTLKASGSFPPEAAHWLCYQAFLLKLAAHSVI
YKCLLGPLRTAQKLLCRKLPEATMTILKAAADPALSTDFQTILD.

In some embodiments, the TERT polypeptide or nucleic acid comprises a human TERT polypeptide or human TERT nucleic acid. In some embodiments, the TERT polypeptide or TERT nucleic acid is non-human. In some embodiments, the TERT polypeptide or TERT nucleic acid is from mouse, horse, dog, rabbit, or goat.

The polypeptides or polynucleotides of the disclosure such as those comprising or encoding for a TERT polypeptide, may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 or more contiguous amino acids or nucleic acids, or any range derivable therein, of SEQ ID Nos:1-10.

The polypeptides or polynucleotides of the disclosure such as those comprising or encoding for a TERT polypeptide, may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, or 2000 or more contiguous amino acids or nucleic acids, or any range derivable therein, of SEQ ID NOS:1-10.

In some embodiments, the polypeptide comprises amino acids or nucleic acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, or 2000 (or any derivable range therein) of SEQ ID NOs:1-10.

In some embodiments, the polypeptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOs: 1-10.

In some embodiments, the polypeptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOs:1-10 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NOS:1-10.

The polypeptides or polynucleotides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or 600 substitutions.

The substitution may be at amino acid position or nucleic acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of one of SEQ ID NOS:1-10.

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 or more amino acids (or any derivable range therein).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other embodiments, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific embodiments, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression. One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### II. Gene Delivery

Certain aspects of the disclosure include administration of a TERT activating therapy to a subject. This may include administration of TERT nucleic acids and/or polypeptides to a subject. The TERT nucleic acids may include a TERT gene, protein, or mRNA encoded on a DNA or RNA. In some embodiments, the methods include administration of DNA encoding for a TERT polypeptide to a subject. In some embodiments, the methods include administration of an RNA encoding a TERT polypeptide to a subject. Techniques pertaining to the transfer of nucleic acids into cells are well-known to those of ordinary skill in the art. Exemplary techniques are discussed below.

### A. Viral Vectors

In certain embodiments, transfer of an expression construct into a cell is accomplished using a viral vector. Techniques using “viral vectors” are well-known in the art. A viral vector is meant to include those constructs containing viral sequences sufficient to (a) support packaging of the expression cassette and (b) to ultimately express a recombinant gene construct that has been cloned therein.

In particular embodiments, the viral vector is a lentivirus vector. Lentivirus vectors have been successfully used in infecting stem cells and providing long term expression.

Another method for delivery of a nucleic acid involves the use of an adenovirus vector. Adenovirus vectors are known to have a low capacity for integration into genomic DNA. Adenovirus vectors result in highly efficient gene transfer.

Adenoviruses are currently the most commonly used vector for gene transfer in clinical settings. Among the advantages of these viruses is that they are efficient at gene delivery to both nondividing and dividing cells and can be produced in large quantities. The vector comprises a genetically engineered form of adenovirus (Grunhaus et al, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. A person of ordinary skill in the art would be familiar with experimental methods using adenoviral vectors.

The adenovirus vector may be replication defective, or at least conditionally defective, and the nature of the adenovirus vector is not believed to be crucial to the successful practice of the invention. The adenovirus may be of any of the 42 different known serotypes or subgroups A-F and other serotypes or subgroups are envisioned. Adenovirus type 5 of subgroup C is the starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector. Adenovirus growth and manipulation is known to those of skill in the art, and exhibits broad host range in vitro and in vivo. Modified viruses, such as adenoviruses with alteration of the CAR domain, may also be used. Methods for enhancing delivery or evading an immune response, such as liposome encapsulation of the virus, are also envisioned. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains two long terminal repeat (LTR) sequences present at the 5′ and 3′ ends of the viral genome. These contain strong promoter and enhancer sequences and are also required for integration in the host cell genome (Coffin, 1990).

In order to construct a retroviral vector, a nucleic acid encoding a nucleic acid or gene of interest is inserted into the viral genome in the place of certain viral sequences to produce a virus that is replication-defective. A person of ordinary skill in the art would be familiar with well-known techniques that are available to construct a retroviral vector.

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al, 1986; Lebkowski et al, 1988; McLaughlin et al, 1988), which means it is applicable for use with the present invention. Details concerning the generation and use of rAAV vectors are described in U.S. Pat. Nos.5,139,941 and 4,797,368, each incorporated herein by reference.

Typically, recombinant AAV (rAAV) virus is made by cotransfecting a plasmid containing the gene of interest flanked by the two AAV terminal repeats (McLaughlin et al, 1988; Samulski et al, 1989; each incorporated herein by reference) and an expression plasmid containing the wild-type AAV coding sequences without the terminal repeats, for example pIM45 (McCarty et al., 1991; incorporated herein by reference). A person of ordinary skill in the art would be familiar with techniques available to generate vectors using AAV virus.

Herpes simplex virus (HSV) has generated considerable interest in treating nervous system disorders due to its tropism for neuronal cells, but this vector also can be exploited for other tissues given its wide host range. Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995). A person of ordinary skill in the art would be familiar with well- known techniques for use of HSV as vectors.

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome.

Other viral vectors may be employed as constructs in the present invention. For example, vectors derived from viruses such as poxvirus may be employed. A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and it has been suggested that VEE may be an extremely useful vector for immunizations (Caley et al., 1997). It is contemplated in the present invention, that VEE virus may be useful in targeting dendritic cells.

A polynucleotide may be housed within a viral vector that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

### B. Nonviral Gene Transfer

Several non-viral methods for the transfer of nucleic acids into cells also are contemplated by certain aspects of the present invention. These include calcium phosphate precipitation (Graham and Van Der Eb, 1973; Chen and Okayama, 1987; Rippe et al, 1990) DEAE-dextran (Gopal, 1985), electroporation (Tur-Kaspa et al, 1986; Potter et al, 1984), nucleofection (Trompeter et al, 2003), direct microinjection (Harland and Weintraub, 1985), DNA-loaded liposomes (Nicolau and Sene, 1982; Fraley et al, 1979) and lipofectamine- DNA complexes, polyamino acids, cell sonication (Fechheimer et al, 1987), gene bombardment using high velocity microprojectiles (Yang et al, 1990), polycations (Boussif et al, 1995) and receptor-mediated transfection (Wu and Wu, 1987; Wu and Wu, 1988). Some of these techniques may be successfully adapted for in vivo or ex vivo use. A person of ordinary skill in the art would be familiar with the techniques pertaining to use of nonviral vectors, and would understand that other types of nonviral vectors than those disclosed herein are contemplated by the present invention. In a further embodiment of the invention, the expression cassette may be entrapped in a liposome or lipid formulation. Liposomes are vesicular structures characterized by a phospholipid bilayer membrane and an inner aqueous medium. Multilamellar liposomes have multiple lipid layers separated by aqueous medium. Also contemplated is a gene construct complexed with Lipofectamine (Gibco BRL). One of ordinary skill in the art would be familiar with techniques utilizing liposomes and lipid formulations.

### C. Lipid-Based Nanovesicles

In some embodiments, a lipid-based nanovesicle, such as a liposome, an exosome, lipid preparations, lipid-based vesicles (e.g., a DOTAP:cholesterol vesicle) are employed in the methods of the disclosure. In some embodiments, the nanovesicle comprising a TERT polypeptide or nucleic acid encoding a TERT polypeptide is administered to the subject. Lipid-based nanovesicles may be positively charged, negatively charged or neutral.

### 1. Liposomes

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes, and multivesicular liposomes. Liposomes provided herein may be positively charged, negatively charged, or neutrally charged. In certain embodiments, the liposomes are neutral in charge.

A multilamellar liposome has multiple lipid layers separated by aqueous medium. Such liposomes form spontaneously when lipids comprising phospholipids are suspended in an excess of aqueous solution. The lipid components undergo self-rearrangement before the formation of closed structures and entrap water and dissolved solutes between the lipid bilayers. Lipophilic molecules or molecules with lipophilic regions may also dissolve in or associate with the lipid bilayer.

In some embodiments, a polypeptide, a nucleic acid, or a small molecule drug may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

A liposome used according to the present embodiments can be made by different methods, as would be known to one of ordinary skill in the art. For example, a phospholipid, such as for example the neutral phospholipid dioleoylphosphatidylcholine (DOPC), is dissolved in tert-butanol. The lipid(s) is then mixed with a polypeptide, nucleic acid, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° and can be used up to three months. When required the lyophilized liposomes are reconstituted in 0.9% saline.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min to 2 h, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

The dried lipids or lyophilized liposomes prepared as described above may be dehydrated and reconstituted in a solution of a protein or peptide and diluted to an appropriate concentration with a suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

Additional liposomes which may be useful with the embodiments of the disclosure include cationic liposomes, for example, as described in WO02/100435A1, U.S. Pat. No. 5,962,016, U.S. Application 2004/0208921, WO03/015757A1, WO04029213A2, U.S. Pat. Nos. 5,030,453, and 6,680,068, all of which are hereby incorporated by reference in their entirety without disclaimer.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040, each incorporated herein by reference.

In certain embodiments, the lipid based nanovesicle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanovesicles of the present embodiments may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes. Phospholipids may be from natural or synthetic sources.

Phospholipids include, for example, phosphatidylcholines, phosphatidylglycerols, and phosphatidylethanolamines; because phosphatidylethanolamines and phosphatidylcholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used.

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), di stearoylphosphatidylcholine (“DSPC”), 1-myristoyl-2-palmitoyl phosphatidylcholine (“WPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DWG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

### 2. Exosomes

The terms “nanovesicle” and “exosomes,” as used herein, refer to a membranous particle having a diameter (or largest dimension where the particles is not spheroid) of between about 10 nm to about 1000 nm, more typically between 30 nm and 1000 nm, and most typically between about 50 nm and 750 nm, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. As used herein, the term “isolated” refers to separation out of its natural environment and is meant to include at least partial purification and may include substantial purification. As used herein, the term “sample” refers to any sample suitable for the methods provided by the present invention. The sample may be any sample that includes exosomes suitable for detection or isolation. Sources of samples include blood, bone marrow, pleural fluid, peritoneal fluid, cerebrospinal fluid, urine, saliva, amniotic fluid, malignant ascites, broncho-alveolar lavage fluid, synovial fluid, breast milk, sweat, tears, joint fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with a disease.

Exosomes may be isolated from freshly collected samples or from samples that have been stored frozen or refrigerated. In some embodiments, exosomes may be isolated from cell culture medium. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One preferred method is differential centrifugation from body fluids or cell culture supernatants. Exemplary methods for isolation of exosomes are described in (Losche et al., 2004; Mesri and Altieri, 1998; Morel et al., 2004). Alternatively, exosomes may also be isolated via flow cytometry as described in (Combes et al., 1997).

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

Isolation of exosomes based on size, using alternatives to the ultracentrifugation routes, is another option. Successful purification of exosomes using ultrafiltration procedures that are less time consuming than ultracentrifugation, and do not require use of special equipment have been reported. Similarly, a commercial kit is available (EXOMIR™ Bioo Scientific) which allows removal of cells, platelets, and cellular debris on one microfilter and capturing of vesicles bigger than 30 nm on a second microfilter using positive pressure to drive the fluid. However, for this process, the exosomes are not recovered, their RNA content is directly extracted from the material caught on the second microfilter, which can then be used for PCR analysis. HPLC-based protocols could potentially allow one to obtain highly pure exosomes, though these processes require dedicated equipment and are difficult to scale up. A significant problem is that both blood and cell culture media contain large numbers of nanoparticles (some non-vesicular) in the same size range as exosomes. For example, some miRNAs may be contained within extracellular protein complexes rather than exosomes; however, treatment with protease (e.g., proteinase K) can be performed to eliminate any possible contamination with “extraexosomal” protein.

### a. Exemplary Protocol for Collecting Exosomes from Cell Culture

On Day 1, seed enough cells (e.g., about five million cells) in T225 flasks in media containing 10% FBS so that the next day the cells will be about 70% confluent. On Day 2, aspirate the media on the cells, wash the cells twice with PBS, and then add 25-30 mL base media (i.e., no PenStrep or FBS) to the cells. Incubate the cells for 24-48 hours. A 48 hour incubation is preferred, but some cells lines are more sensitive to serum-free media and so the incubation time should be reduced to 24 hours. Note that FBS contains exosomes that will heavily skew NanoSight results.

On Day 3/4, collect the media and centrifuge at room temperature for five minutes at 800×g to pellet dead cells and large debris. Transfer the supernatant to new conical tubes and centrifuge the media again for 10 minutes at 2,000×g to remove other large debris and large vesicles. Pass the media through a 0.2 μm filter and then aliquot into ultracentrifuge tubes (e.g., 25×89 mm Beckman Ultra-Clear) using 35 mL per tube. If the volume of media per tube is less than 35 mL, fill the remainder of the tube with PBS to reach 35 mL. Ultracentrifuge the media for 2-4 hours at 28,000 rpm at 4° C. using a SW 32 Ti rotor (k-factor 266.7, RCF max 133,907). Carefully aspirate the supernatant until there is roughly 1-inch of liquid remaining. Tilt the tube and allow remaining media to slowly enter aspirator pipette. If desired, the exosomes pellet can be resuspended in PBS and the ultracentrifugation at 28,000 rpm repeated for 1-2 hours to further purify the population of exosomes.

Finally, resuspend the exosomes pellet in 210 μL PBS. If there are multiple ultracentrifuge tubes for each sample, use the same 210 μL PBS to serially resuspend each exosomes pellet. For each sample, take 10 μL and add to 990 μL H₂O to use for nanoparticle tracking analysis. Use the remaining 200 μL exosomes-containing suspension for downstream processes or immediately store at −80° C.

### b. Exemplary Protocol for Extracting Exosomes from Serum Samples

First, allow serum samples to thaw on ice. Then, dilute 250 μL of cell-free serum samples in 11 mL PBS; filter through a 0.2 μm pore filter. Ultracentrifuge the diluted sample at 150,000×g overnight at 4° C. The following day, carefully discard the supernatant and wash the exosomes pellet in 11 mL PBS. Perform a second round of ultracentrifugation at 150,000×g at 4° C. for 2 hours. Finally, carefully discard the supernatant and resuspend the exosomes pellet in 100 μL PBS for analysis.

### c. Exemplary Protocol for Electroporation of Exosomes and Liposomes

Mix 1×10⁸ exosomes (measured by NanoSight analysis) or 100 nm liposomes (e.g., purchased from Encapsula Nano Sciences) and 1 μg of siRNA (Qiagen) or shRNA in 400 μL of electroporation buffer (1.15 mM potassium phosphate, pH 7.2, 25 mM potassium chloride, 21% Optiprep). Electroporate the exosomes or liposomes using a 4 mm cuvette (see, e.g., Alvarez-Erviti et al., 2011; El-Andaloussi et al., 2012). After electroporation, treat the exosomes or liposomes with protease-free RNase followed by addition of 10× concentrated RNase inhibitor. Finally, wash the exosomes or liposomes with PBS under ultracentrifugation methods, as described above.

### d. Administration of Therapeutic Exosomes

Certain aspects of the disclosure provide for treating a patient with exosomes that express or comprise a therapeutic agent, such as a TERT polypeptide or nucleic acid. As exosomes are known to comprise the machinery necessary to complete mRNA transcription and protein translation (see PCT/US2014/068630, which is incorporated herein by reference in its entirety), mRNA or DNA nucleic acids encoding a therapeutic protein may be transfected into exosomes. Alternatively, the therapeutic protein itself may be electroporated into the exosomes or incorporated directly into a liposome. In some embodiments, the exosome further comprises an additional therapeutic agent, such as a therapeutic agent described herein. Provided herein are methods and drugs that use engineered liposomes and exosomes as delivery systems for treatment of disease.

### 3. CD47-Expressing Nanovesicles

In some embodiments, pharmaceutical compositions are provided that comprise a lipid-based nanovesicle comprising CD47 on its surface and wherein the lipid-based nanovesicle comprises a TERT polypeptide or a nucleic acid encoding for a TERT polypeptide.

In some aspects, the lipid-based nanoparticle is a liposome or an exosome. In certain aspects, the exosomes are isolated from cells over-expressing CD47. In some aspects, the exosomes are isolated from a patient in need of treatment. In some aspects, the exosomes are isolated from fibroblasts. In some aspects, the liposome is a single lamellar liposome. In some aspects, the liposome is a multilamellar liposome.

In some aspects, the composition is formulated for parenteral administration, such as, for example, intravenous, intramuscular, sub-cutaneous, or intraperitoneal injection.

In some aspects, the composition comprises an antimicrobial agent. The antimicrobial agent may be benzalkonium chloride, benzethonium chloride, benzyl alcohol, bronopol, centrimide, cetylpyridinium chloride, chlorhexidine, chlorobutanol, chlorocresol, chloroxylenol, cresol, ethyl alcohol, glycerin, exetidine, imidurea, phenol, phenoxyethanol, phenylethl alcohol, phenlymercuric nitrate, propylene glycol, or thimerosal.

In some aspects, a single lipid-based nanovesicle comprises more than one agent, such as a TERT polypeptide or nucleic acid and one or more additional therapeutic agents described herein.

In one embodiment, methods are provided for administering a TERT activating therapy to a patient, wherein the TERT activating therapy therapeutic comprises exosomes. In some aspects, the disclosure relates to transfecting exosomes with a nucleic acid (e.g., a DNA or an RNA) encoding a TERT polypeptide, incubating the transfected exosomes under conditions to allow for expression of TERT within the exosomes, and providing the incubated exosomes to the patient, thereby administering TERT activating therapy to the patient.

### III. Administration of Therapeutic Compositions

The therapy provided herein may comprise administration of a combination of therapeutic agents, such as a first TERT activating therapy and a second therapy. The therapies may be administered in any suitable manner known in the art. For example, the first and second treatment may be administered sequentially (at different times) or concurrently (at the same time). In some embodiments, the first and second therapies are administered in a separate composition. In some embodiments, the first and second therapies are in the same composition. In some embodiments, methods and compositions of the disclosure comprise administration of an additional therapy. In some embodiments, the additional therapy comprises a cholinesterase inhibitor such as donepezil, galantamine, or rivastigmine. In some embodiments, the additional therapy comprises memantine.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed, for example, a first treatment is “A” and a second treatment is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

The therapeutic agents of the disclosure may be administered by the same route of administration or by different routes of administration. In some embodiments, the therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein).

In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IV. Treatment of Disease

The methods of the disclosure may be used to treat or prevent certain age-related diseases, conditions, or disorders. Non-limiting examples of age-related diseases, conditions, or disorders include insulin resistance (i.e., impaired glucose tolerance), benign prostatic hyperplasia, hearing loss, osteoporosis, age-related macular degeneration, neurodegenerative diseases, a skin disease, aging skin, or cancer. Non-limiting examples of neurodegenerative diseases include Alzheimer disease; epilepsy; Huntington's Disease; Parkinson's Disease; stroke; spinal cord injury; traumatic brain injury; Lewy body dementia; Pick's disease; Niewmann-Pick disease; amyloid angiopathy; cerebral amyloid angiopathy; systemic amyloidosis; hereditary cerebral hemorrhage with amyloidosis of the Dutch type; inclusion body myositis; mild cognitive impairment; Down's syndrome; and neuromuscular disorders including amyotrophic lateral sclerosis (ALS), multiple sclerosis, and muscular dystrophies including Duchenne dystrophy, Becker muscular dystrophy, Facioscapulohumeral (Landouzy-Dejerine) muscular dystrophy, and limb-girdle muscular dystrophy (LGMD). Also included is neurodegenerative disease due to stroke, head trauma, spinal injury, or other injuries to the brain, peripheral nervous, central nervous, or neuromuscular system.

Certain embodiments of the methods set forth herein pertain to methods of preventing a disease or health-related condition in a subject. Preventive strategies are of key importance in medicine today.

In some embodiments, the treatment is for the premature aging or a disease associated with premature aging. Examples of premature aging disorders include Hutchinson-Gilford progeria syndrome (HGPS), Néstor-Guillermo progeria syndrome, Werner syndrome, Cockayne syndrome, Bloom syndrome, xeroderma pigmentosum, ataxia telangiectasia, trichothiodystrophy, dyskeratosis congenital, and mosaic variegated aneuploidy syndrome. In some embodiments, one or more of premature aging disease, disease associated with premature aging, age-related disease, neurodegenerative disease, or disorders described herein is excluded from the methods of the disclosure.

### V. Kits

Certain aspects concern kits containing compositions described herein or compositions to implement methods described herein.

In various aspects, a kit is envisioned containing therapeutic agents and/or other therapeutic and delivery agents. In some embodiments, a kit for preparing and/or administering a therapy described herein may be provided. The kit may comprise one or more sealed vials containing any of the pharmaceutical compositions, therapeutic agents and/or other therapeutic and delivery agents. In some embodiments, the lipid is in one vial, and the therapeutic agent is in a separate vial. The kit may include, for example, at least one TERT activating therapy, one or more lipid component, as well as reagents to prepare, formulate, and/or administer the components described herein or perform one or more steps of the methods. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure of delivering a pharmaceutically effective amount of a therapeutic agent.

In some embodiments, kits may be provided to evaluate the expression of TERT or related molecules. Such kits can be prepared from readily available materials and reagents. For example, such kits can comprise any one or more of the following materials: enzymes, reaction tubes, buffers, detergent, primers and probes, nucleic acid amplification, and/or hybridization agents. In a particular embodiment, these kits allow a practitioner to obtain samples in blood, tears, semen, saliva, urine, tissue, serum, stool, colon, rectum, sputum, cerebrospinal fluid and supernatant from cell lysate. In another embodiment, these kits include the needed apparatus for performing RNA extraction, RT-PCR, and gel electrophoresis. Instructions for performing the assays can also be included in the kits.

Kits may comprise components, which may be individually packaged or placed in a container, such as a tube, bottle, vial, syringe, or other suitable container means. The components may include probes, primers, antibodies, arrays, negative and/or positive controls. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

The kit can further comprise reagents for labeling TERT in the sample. The kit may also include labeling reagents, including at least one of amine-modified nucleotide, poly(A) polymerase, and poly(A) polymerase buffer. Labeling reagents can include an amine-reactive dye or any dye known in the art.

The components of the kits may be packaged either in aqueous media or in lyophilized form. The container means of the kits will generally include at least one vial, test tube, flask, bottle, syringe or other container means, into which a component may be placed, and preferably, suitably aliquotted. Where there is more than one component in the kit (labeling reagent and label may be packaged together), the kit also will generally contain a second, third or other additional container into which the additional components may be separately placed. However, various combinations of components may be comprised in a vial. The kits may also include a means for containing the nucleic acids, antibodies or any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

Alternatively, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits in certain aspects. The dye may then be resuspended in any suitable solvent, such as DMSO.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits may include a means for containing the vials in close confinement for commercial sale, such as, e.g., injection and/or blow-molded plastic containers into which the desired vials are retained.

A kit may also include instructions for employing the kit components as well the use of any other reagent not included in the kit. Instructions may include variations that can be implemented.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Identification of Telomerase Activation as a Therapeutic Strategy for Alleviating Alzheimer's Pathology Using Novel Inducible TERT-AD Mouse Model

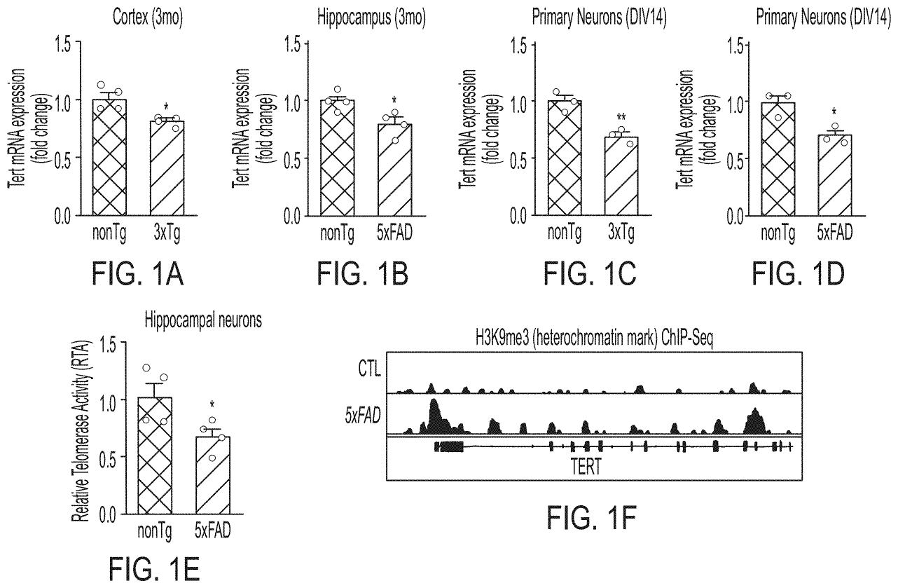

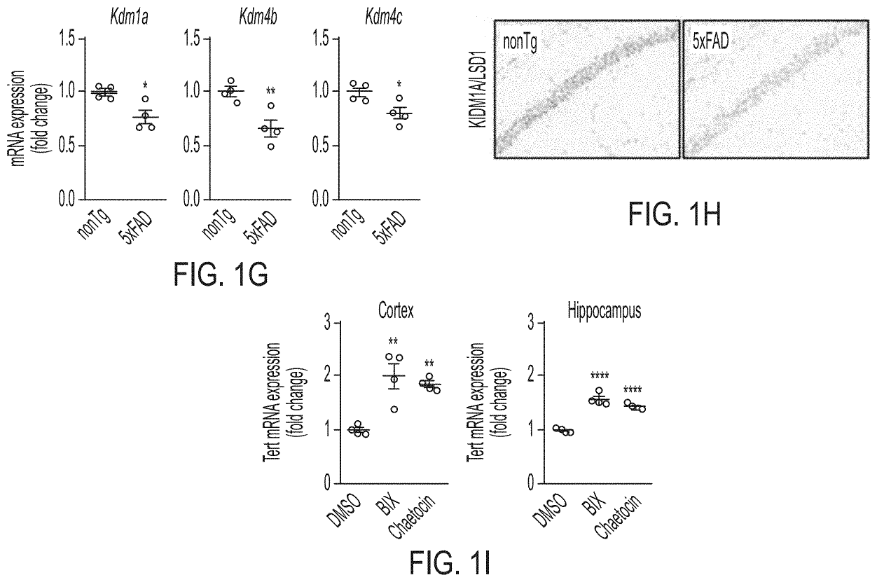

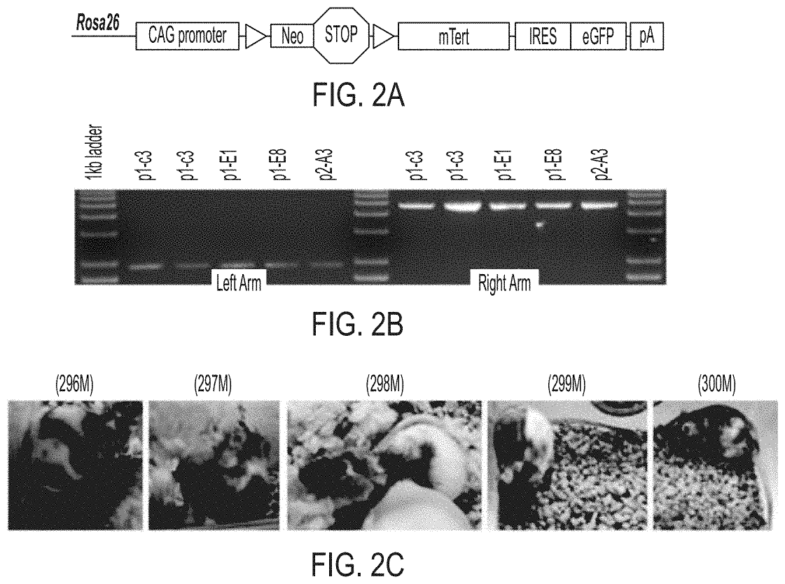

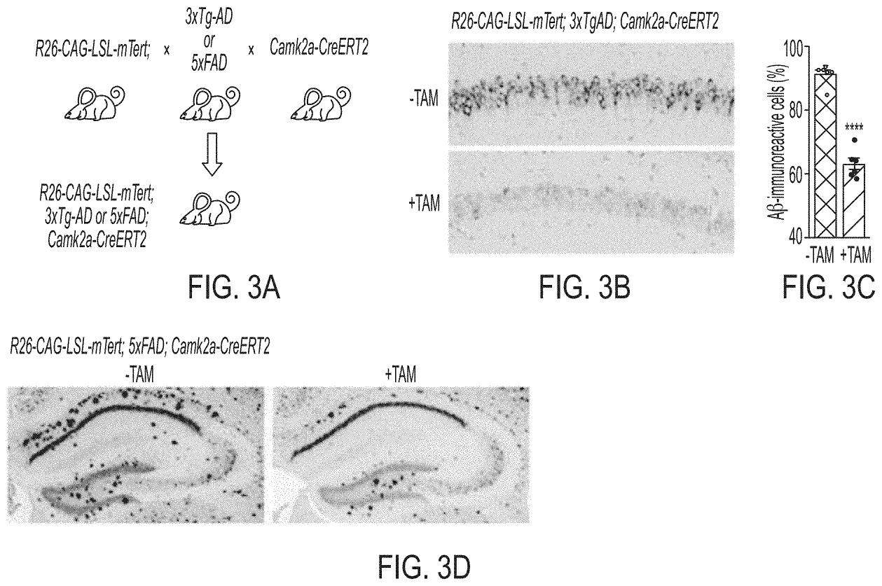

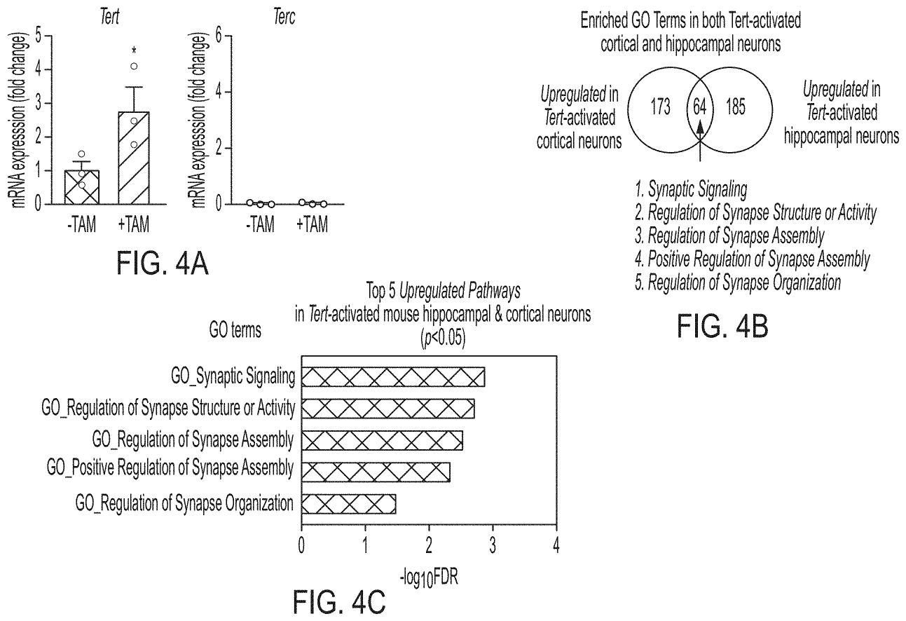

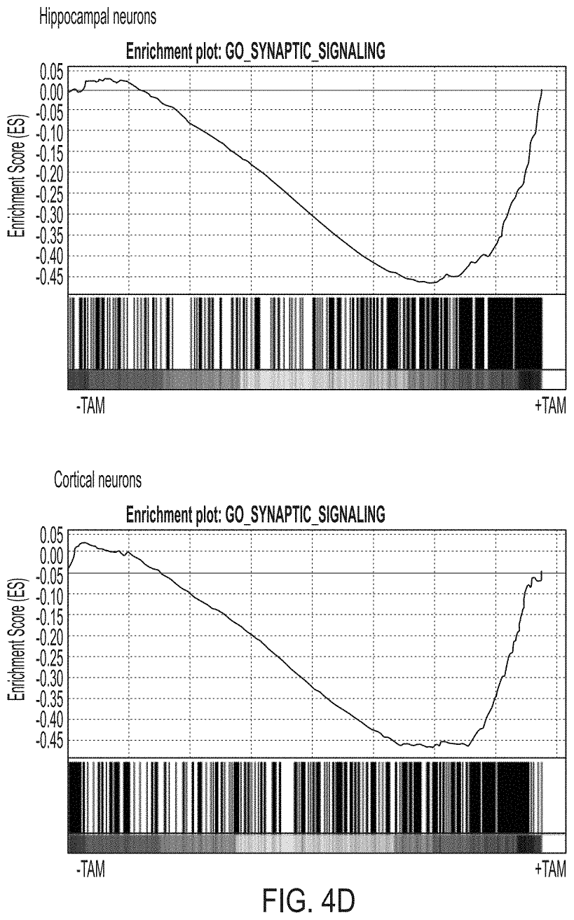

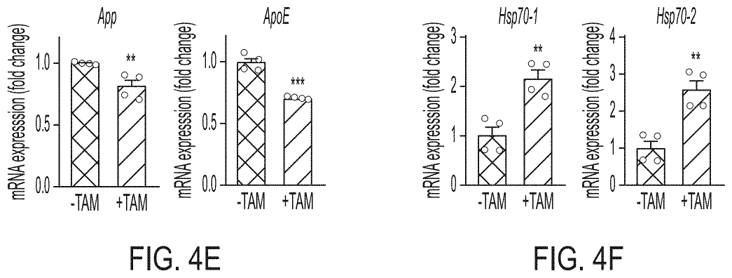

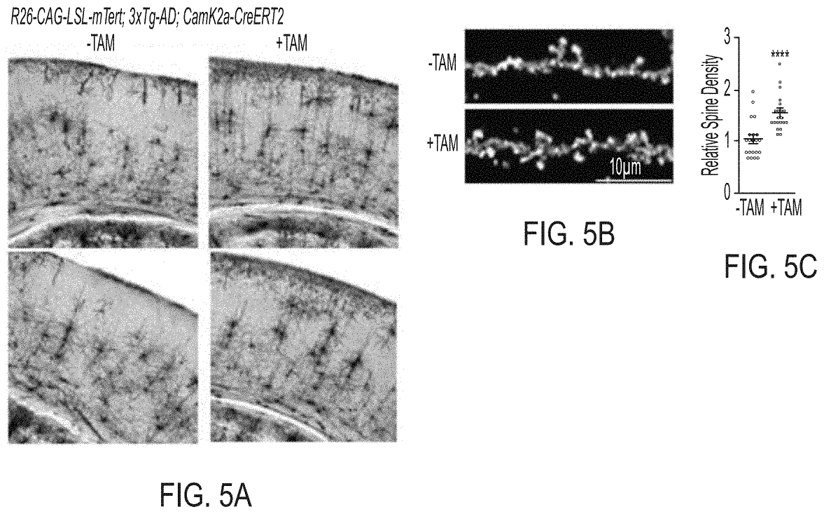

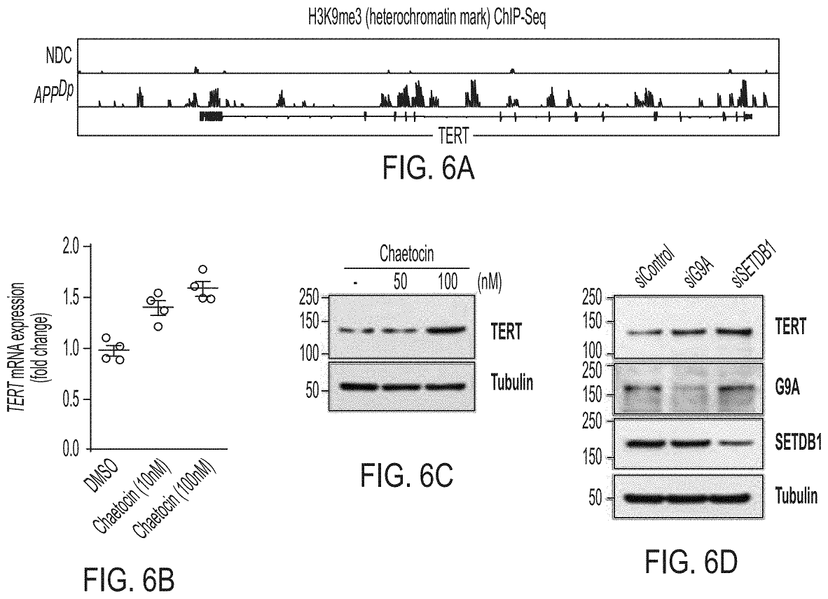

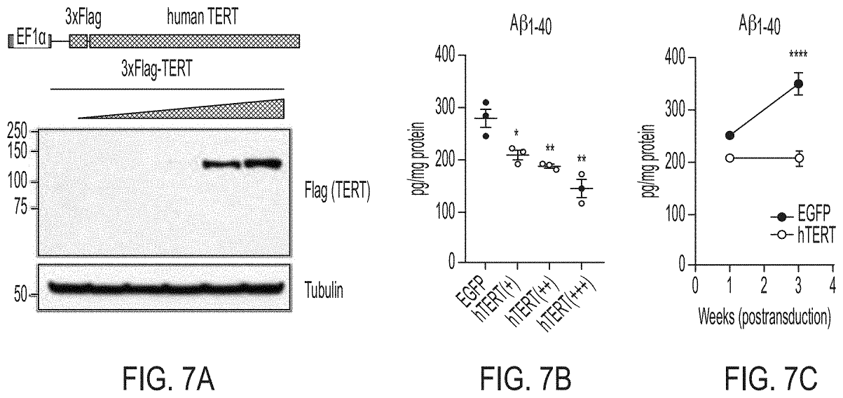

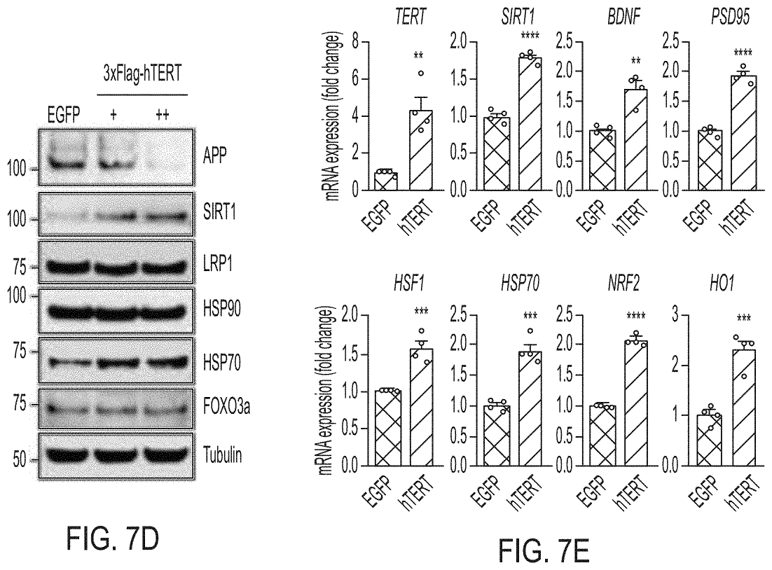

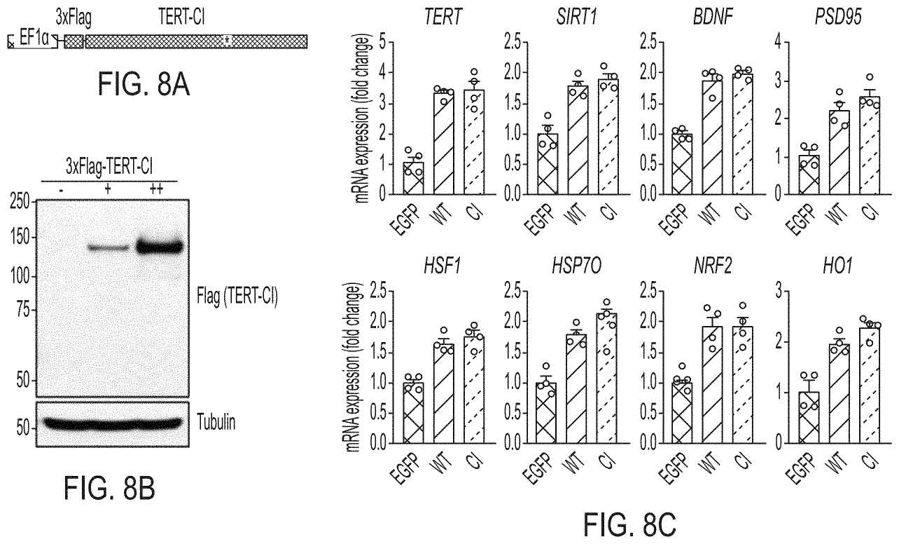

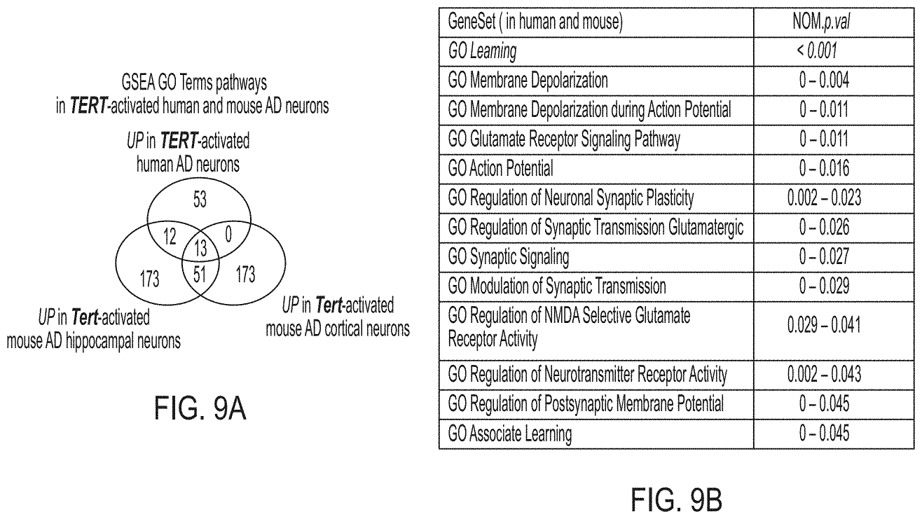

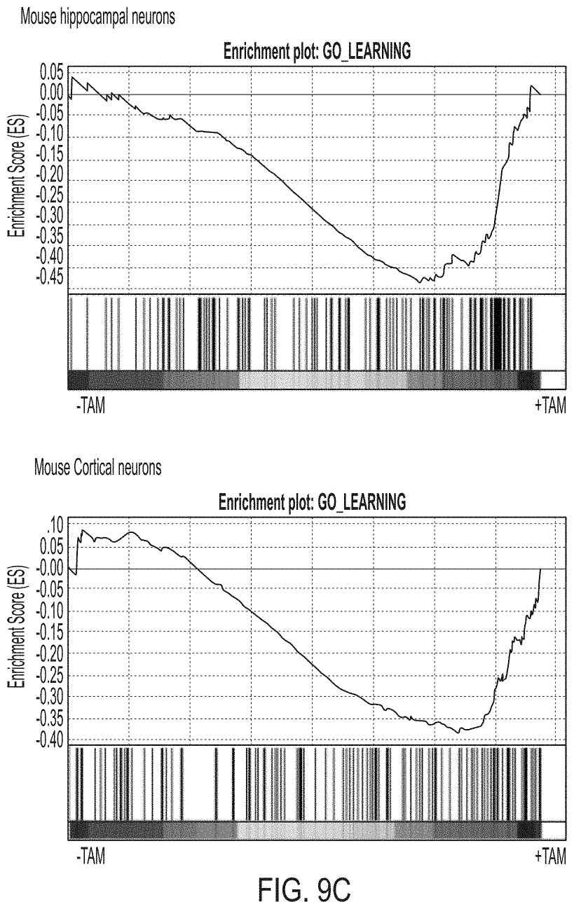

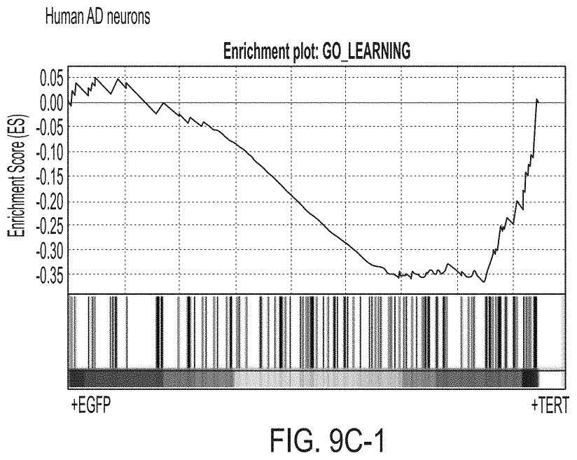

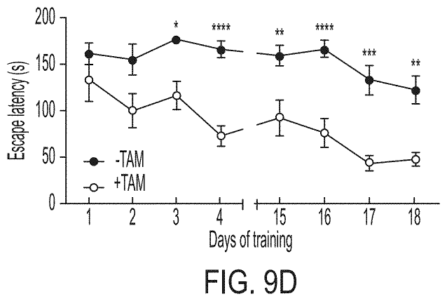

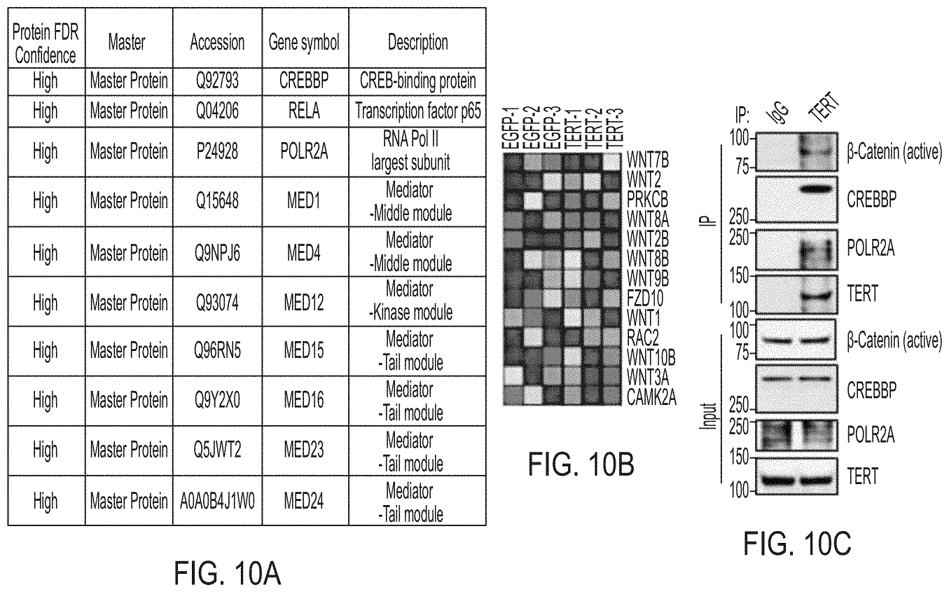

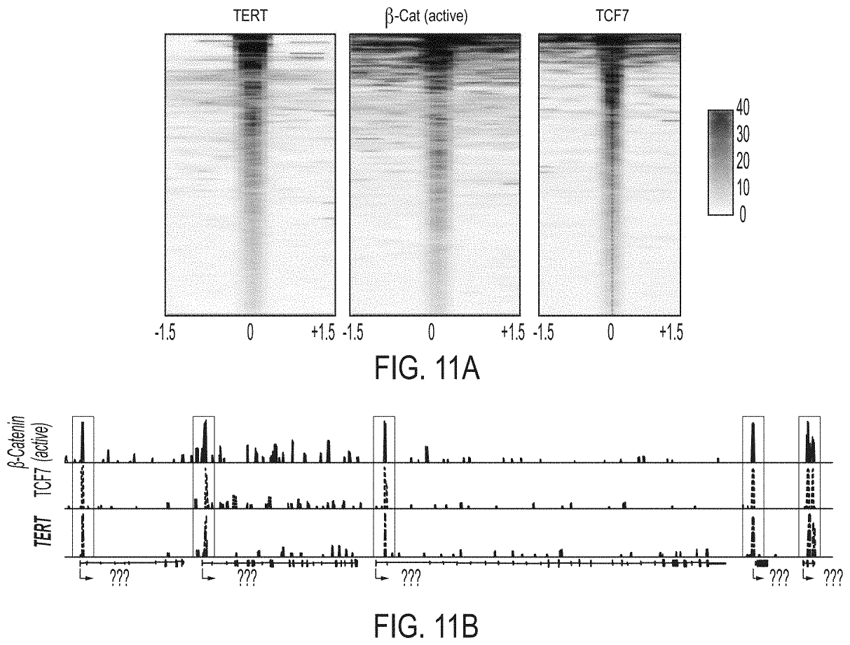

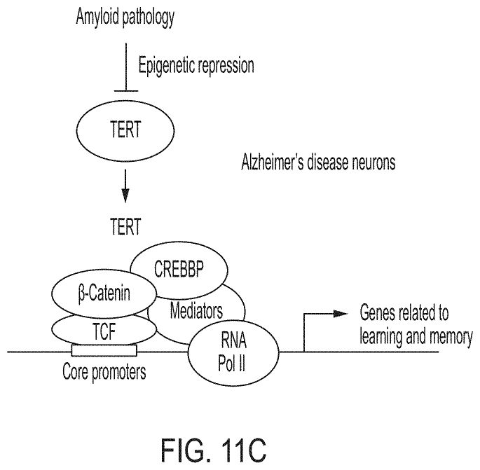

The inventors observed that Tert gene expression was significantly downregulated in brain tissues of two distinct and well-established mouse models of AD, 3xTg-AD developing both amyloid and tau pathologies (Oddo et al., Neuron, 2003), 5xFAD only developing amyloid pathology (Oakley et al., J Neurosci, 2006) in 3-month-old mice, which exhibit elevated levels of AP yet minimal signs of neurodegeneration (FIG. 1A,B). Focusing in particular on the neuronal population, the inventors isolated and cultured primary cortical and hippocampal neurons from E18.5-E19.5 AD mouse brains, and Tert mRNA levels were examined at 14 Days in vitro (DIV), by which time the synaptic network is mature. Consistent with previous results from in vivo mouse brain tissues, Tert expression was downregulated in both 3xTg-AD and 5xFAD primary neurons relative to wildtype controls (FIG. 1C,D). Correspondingly, telomerase activity was also lower in freshly isolated hippocampal neurons from 5xFAD brain relative to wildtype controls (FIG. 1E). More interestingly, the inventors observed the high occupancy of repressive epigenetic mark, H3K9me3, which has been known to be mainly accumulated at gene bodies and critical for gene repression in neuronal genes, in the Tert gene body and promoter region in 5xFAD mouse neurons (FIG. 1F). Histone methylation is reversible and histone demethylases mediate the removal of methyl groups from lysine residues on histones (Greer and Shi, Nat Rev Genet, 2012). Interestingly, the inventors examined the levels of histone methyltransferases and demethylases and revealed that H3K9 demethylases Kdm1a, Kdm4b and Kdm4c were significantly downregulated in the cortical and hippocampal neurons of the mouse AD brains relative to wildtype controls (FIG. 1G,H). To investigate whether reversible H3K9 methylation is involved in the repression of Tert, the inventors assessed the impact of histone methyltransferase inhibitors chaetocin and BIX-01294, the non-selective cofactor-competitive inhibitor and the selective substrate-competitive inhibitor, respectively, in the AD mouse model (Greiner et al., Nat Chem Biol, 2005; Kubicek et al., Mol Cell, 2007; Yuan et al., ACS Chem Biol, 2012). The peripheral administration of these compounds has been documented to reduce H3K9 methylation marks in the central nervous system (Dixit et al., Cell Death Dis, 2014; Chase et al., PLoS One, 2019). Both small-molecule histone methyltransferase inhibitors resulted in de-repression of Tert gene expression in the cortex and hippocampus of AD mice (FIG. 1I). Together with previous work showing that this epigenetic mark accumulates at gene bodies and is critical for transcriptional repression of neuronal genes (Liu et al., J Neurosci, 2015), the inventors confirmed the possibility that soluble Aβ may negatively regulate Tert gene expression via altered expression of H3K9 demethylases or methyltransferases in AD mouse neurons.

Given the repression of Tert gene expression early in the accumulation of amyloid in the AD mouse models, the inventors tested whether increased Tert gene expression in AD neurons could ameliorate or prevent amyloid pathophysiology. To that end, the inventors generated Cre-inducible Tert knock-in allele that consists of the ubiquitously expressed CAG promoter, followed by the loxP-flanked stop cassette and mouse Tert open reading frame (R26-CAG-LSL-mTert). The linearized construct was targeted into the Rosa 26 locus of C57BL/6-derived JM8F6 embryonic stem (ES) cells by electroporation (FIG. 2A). The inventors identified positive clones by Long Range PCR (New England Biolabs) using the following primers: left arm 5′-GGT CGT GTG GTT CGG TGT CTC TTT-3′ and 5′-ATG GGC TAT GAA CTA ATG ACC CCG-3′ right arm 5′- CAC TAC CAG CAG AAC ACC CCC ATC-3′ and 5′-GTG CCA CTA GTA CCA ACA GCC TCT-3′ (FIG. 2B). The inventors confirmed the correct recombination by sequencing and karyotyping. Eventually, the inventors identified two independent clones and injected into C57BL/6 albino blastocysts to generate chimeric mice, and the chimeric mice from each clone was able to produce germline transmission (FIG. 2C).

In order to further investigate the role of telomerase activation in AD mouse model, the inventors first crossed this new Cre-inducible Tert knock-in allele with 3xTg-AD or 5xFAD. Subsequently, to selectively drive Tert expression in neuronal populations of the AD mouse models, the inventors incorporated a neuron-specific Cre allele which is under the control of the calcium/calmodulin-dependent protein kinase type II alpha promoter (Camk2a-CreERT2) (Madisen et al., Nat Neurosci, 2010). The inventors successfully established both R26-CAG-LSL-mTert; 3xTg-AD; Camk2a-CreERT2 and R26-CAG-LSL-mTert; 5xFAD; Camk2a-CreERT2 strains which result in deletion of the floxed stopper sequences following tamoxifen administration, leading to turning on of mTert gene expression in the neurons of each AD mouse strain (FIG. 3A). These models enabled spatial (neuron-specific) and temporal (tamoxifen-inducible) control of Tert gene expression in two independent and widely studied AD (3xTg-AD and 5xFAD) mouse models.

To examine the potential impact of telomerase activation on AD pathology in vivo, the inventors treated R26-CAG-LSL-mTert; 3xTg-AD; Camk2a-CreERT2 with tamoxifen at 2˜3 months of age, at which time intracellular and cytotoxic Aβ oligomers begin to accumulate in the brain, and evaluated the effect of enhanced Tert expression on amyloid pathology. The inventors revealed a striking decline in Aβ deposition in the hippocampus of Tert-activated R26-CAG-LSL-mTert; 3xTg-AD; Camk2a-CreERT2 mouse model (FIG. 3B,C). Similar amyloid load reduction was observed in the R26-CAG-LSL-mTert; 5xFAD; Camk2a-CreERT2 model (FIG. 3D).

The inventors next investigated the molecular mechanism driving a reduction in amyloid plaque burden. To achieve a comprehensive understanding of Tert's role in neuronal populations, the inventors performed genome-wide RNA sequencing (RNA-Seq) analysis. The inventors examined R26-CAG-LSL-mTert; 3xTg-AD; Camk2a-CreERT2 mice with tamoxifen to induce Tert expression in AD neurons in vivo, and cortical and hippocampal neurons were separately isolated from adult mouse brain after tamoxifen treatment to probe early transcriptional responses of telomerase activation. This profile confirmed increased Tert gene expression in isolated neurons of our model upon tamoxifen treatment, and no changes in expression of Terc gene which encodes the telomerase RNA component (FIG. 4A). Computational analysis revealed that Tert induction in cortical and hippocampal AD neurons correlated with activation of multiple signaling pathways associated with synaptic signaling, regulation of synapse structure or activity, regulation of synapse assembly, positive regulation of synapse assembly, and regulation of synapse organization (FIG. 4B,C). Gene set enrichment analysis (GSEA) also showed that genes involved in synaptic signaling were globally upregulated in both neuronal populations after Tert induction (FIG. 4D). The inventors also examined the expression of genes integral to AD biology. Strikingly, the inventors found that the expression of App (amyloid-β precursor protein) and ApoE (apolipoprotein E, a strong genetic risk factor for AD) genes were significantly reduced in Tert-activated AD neurons (FIG. 4E). Simultaneously, the gene expression of Hsp70, a molecular chaperone which can reduce the Aβ-induced cellular toxicity and has been shown to effectively protect neurons in various AD animal models, was notably induced under Tert induction (FIG. 4F). By utilizing these unbiased transcriptomic analyses, the inventors identified that Tert induction in neurons can impact the expression of a large group of genes in postmitotic neurons in vivo that are strongly linked to AD pathobiology and central to synapse formation and neuronal activity.

Synapse loss and dysfunction are major correlates of cognitive decline in AD (Palop and Mucke, Nat Neurosci, 2010; Hong et al., Science, 2016; Selkoe and Hardy, EMBO Mol Med, 2016). To test whether induction of neuronal Tert could lead to protection against synaptic and network dysfunction in the AD brain, the inventors examined neuronal morphology in vivo using Golgi-Cox staining. The inventors observed that Tert induction was associated with increased neuronal complexity and density of dendritic spines in the aged cerebral cortex of Tert-activated R26-CAG-LSL-mTert; 3xTg-AD; Camk2a-CreERT2 mice relative to controls (FIG. 5A,B,C). The inventors conclude that elevated Tert expression in neurons activates synaptic signaling cascades and reduces spine shrinkage and synaptic loss in neurons of the mouse AD brain.

Together with these murine observations, the inventors also sought to assess the biological impact of TERT induction in the context of human AD. The inventors employed well-established induced pluripotent stem cells (iPSCs) which were derived from familial AD patient harboring genomic duplication of APP gene (APP^{DP}) (Israel et al., Nature, 2012). Consistent with inventors' findings from the murine models, the inventors found that human AD neurons derived from APP^{DP} patient also had high occupancy of the repressive epigenetic mark H3K9me3 in TERT gene body relative to non-demented control (FIG. 6A). To further probe the requirement of H3K9 methylation for human TERT expression, the inventors next investigated the effect of inhibition of H3K9 methyltransferases in human AD neurons. Consistent with the murine in vivo findings (FIG. 1I), inhibiting H3K9 methylation also restored both TERT mRNA and protein expression in human AD neurons (FIG. 6B,C,D).

To examine whether TERT activation can also impact Aβ pathology in human contexts, the inventors generated lentiviral human TERT construct under EF1a promoter, and measured the impact of TERT induction on Aβ accumulation in differentiated human AD neurons infected with lentiviral vectors expressing TERT or EGFP (FIG. 7A). Similar to the murine studies, the inventors found that TERT induction resulted a significant dose- and time-dependent reduction in intracellular Aβ accumulation in human AD neurons as measured by sandwich ELISA (enzyme-linked immunosorbent assay) (FIG. 7B,C). To further understand the underlying mechanisms of TERT-mediated attenuation of amyloid load in neurons, the inventors sought to identify possible molecular targets. In addition to lessening Aβ accumulation, TERT induction not only decreased APP protein levels, but also triggered activation of the anti-aging gene (SIRT1), molecular chaperone and stress sensor genes (HSP70 and HSF1), synaptic plasticity-related genes (BDNF and PSD-95), and antioxidant genes (NRF2 and HO1) (FIG. 7D,E), which are known to be crucial in reducing Aβ processing and cytotoxicity as well as improving synaptic plasticity and memory formation in the adult brain (Evans et al., J Biol Chem, 2006; Qin et al., J Biol Chem, 2006; Herskovits and Guarente, Neuron, 2014; Lackie et al., Front Neurosci-Switz, 2017). The inventors' findings suggest that TERT activation can not only diminish Aβ production, but exert neuroprotective actions in AD neurons through the production of neuroprotective mediators.

To further ascertain whether catalytic activity is required for TERT-dependent gene regulation at the transcription level, the inventors generated catalytically inactive (CI) TERT expression construct by substituting the aspartic acid at position 712 residue (Weinrich et al., Nat Genet, 1997) with the alanine using site-directed mutagenesis (FIG. 8A,B). The inventors unveiled that the catalytically inactive TERT mutant also led to the upregulation of these genes (FIG. 8C), indicating that TERT's transactivation function is independent of its catalytic activity.

To gain insight into the functional significance of neuronal TERT activation in AD, the inventors intersected RNA-seq transcriptional profiles and pathway analysis of mouse AD cortical neurons, mouse AD hippocampal neurons, and human AD neurons. Using this integrative cross-species analysis of the neuronal TERT activation network, the inventors identified overlap of multiple neuron-specific pathways (FIG. 9A) with learning process as the most significantly enriched pathway (all p<0.001) followed by membrane depolarization, glutamate receptor signaling, action potential, and synaptic signaling as the downstream consequences of TERT activation (FIG. 9B). The inventors also found that all the enrichment profiles from three groups displayed a high level of concordant regulation of genes sets involved in learning processes in mouse and human AD neurons (FIG. 9C), suggesting that TERT regulates critical disease-associated pathways in the AD brain.

As TERT induces dendritic spine formation on the cellular and tissue levels as well as activates learning process genes on the molecular level, the inventors next investigated whether TERT activation could ameliorate the learning deficits of AD models in vivo. To that end, spatial learning and memory were assessed in the R26-CAG-LSL-mTert; 3xTg-AD; Camk2a-CreERT2 model versus AD controls. While AD controls showed impaired acquisition of spatial learning at old age on the Barnes maze, age- and gender-matched Tert-activated AD mice achieved significant improvement in learning ability and memory retention, as indicated by a reduction in latencies to enter the escape hole (FIG. 9D). Aligning with above cellular and molecular data, the inventors' findings indicate that Tert activation attenuates age-associated learning impairment of AD mice.

The inventors further investigated the mechanistic details underlying TERT's role in terminally differentiated postmitotic neurons. To identify the mechanistic basis of TERT activation and gene regulation, the inventors conducted proteome-wide analysis of potential interaction partners of TERT in neurons. Characterization of TERT-containing protein complexes by mass spectrometry identified transcriptional regulators CREB-binding protein (CREBBP) and RELA, RNA polymerase II largest and catalytic subunit POLR2A, and multiple Mediator complex subunits (MED 1, 4, 12, 15, 16, 23, 24) which link transcriptional regulators to RNA polymerase II in human neurons (FIG. 10A). The inventors also revealed that various WNTs and WNT pathway components were elevated in TERT-activated AD neurons by RNA-Seq analysis (FIG. 10B) which gains added significance in light of the known neuroprotective action of WNT signaling in neurodegenerative disease. Based on these observations, the inventors assessed whether endogenous TERT in postmitotic neurons physically interacts with transcriptional regulatory complexes containing β-Catenin, a pivotal player in the transduction of WNT signaling. Co-immunoprecipitation assay indeed confirmed that neuronal TERT protein physically interacts with the activated nuclear form of β-Catenin as well as CREBBP and POLR2A at endogenous levels in fully differentiated human neurons (FIG. 10C).

The inventors further assessed a possible global enrichment of the association of TERT and β-Catenin/TCF7 on the genomic level. The inventors determined the genome-wide distribution of TERT and β-Catenin/TCF7 in human neurons by ChIP-Seq using specific antibodies, and discovered that both TERT and β-Catenin as well as TCF7, which is transcription complex partner, predominantly occupied the transcription start sites (TSS) of gene promoters in human neurons (FIG. 11A). The inventors also defined that TERT-binding sites were occupied by both β-Catenin and TCF7 at the promoter regions of highly relevant genes including a WNT family member WNT9B, a Na⁺/K⁺-ATPase catalytic subunit ATP1A3 (one of 5 overlapping genes upregulated in both TERT-activated human and mouse neurons in our study), HSP70 family members HSPA12A and HSPA6, and a positive feed-forward regulator of TERT, MYC (FIG. 11B). The inventors' findings of the physical association of TERT and the β-Catenin/TCF transcription complex and the TERT enhancement of β-Catenin/TCF transcriptional activity in AD neurons (FIG. 11C) point to important roles for TERT and WNT signaling in the progression of AD disease.

In this invention, the inventors identified that murine and human neurons from amyloid-based AD models exhibit epigenetic repression of neuronal TERT expression, prompting exploration of the relationship between amyloid accumulation and TERT gene expression and whether restoration of TERT expression could impact the disease trajectory. The inventors observed that TERT activation results in a marked reduction of Aβ levels in hippocampal and cortical neurons in the brains of two AD mouse models and in cultured human iPSC-derived AD neurons harboring genomic APP duplication. Mechanistically, TERT induced gene expression and physically interacted with core transcriptional and β-Catenin/TCF7 complex components at the transcriptional start sites of key neuronal genes governing synaptic signaling and learning pathways and protecting neuron health in both mouse and human neurons. Neuronal TERT expression improved dendritic spine formation and cognitive function in aged AD mouse models. Together, these findings support the development of somatic TERT activation therapy as a potential disease modification strategy for AD.

### Example 2—Exosome-Mediated Delivery of TERT mRNA in Alzheimer's Disease Brain

Exosomes are extracellular small vesicles (40˜100 nM) that are released from cells and found in most biological fluids, and provide a useful means of transmission of macromolecules, such as nucleic acids and proteins, into target cells. Exosome therapies have been explored in anti-cancer clinical trials and can be also used to treat neurodegenerative diseases due to their ability to cross the blood-brain barrier easily, while liposomes are preferentially degraded by enzymes, mechanical strain and/or phagocytic attacks before they are delivered to the target sites. The display of CD47 and RVG brain-targeting peptide on the surface of exosomes may not only increase the biological stability by protecting themselves from degradation, but also improve the overall delivery efficiency of bioactive exosomal nucleic acids to target cells in the brain, when compared to liposomes. Targeted exosomes exhibiting a superior ability to deliver TERT mRNA to the brains can serve as effective therapeutic strategies for AD treatment.

### A. Procedures

### 1. Cell Preparation for Exosome Generation

Human fibroblasts and/or bone marrow dendritic cells (BMDCs) can be used as a source of exosomes. These cells can be cultured in DMEM supplemented with 10% exosome-depleted FBS and 1% penicillin-streptomycin.

### 2. Generation of Targeted Exosomes by Display of RVG Brain-Targeting Peptide and CD47 ‘Don't Eat Me’ Signal

The cells can be transfected with plasmids encoding CD47 and RVG (rabies virus glycoprotein)-derived peptide using X-tremeGENE transfection reagents (Roche) or Lipofectamine 2000 reagents (Invitrogen). CD47 ligand protein may interact with signal-regulatory protein α (SIRPα), then initiating a ‘don't eat me’ signal that may protect the exosomes from phagocytosis. RVG-derived peptide on the exosome surface target may guide exosomes to bind to neuronal cells expressing acetylcholine receptors and allow transvascular delivery of targeted exosomes to the central nervous system.

### 3. Isolation of Targeted Exosomes by Microfiltration and Ultracentrifugation

Targeted exosomes can be purified by differential centrifugation steps. The supernatant supplemented with exosome-depleted FBS can be collected from cells, filtered using 0.2-μm filters, ultra-centrifuged at 120,000×g for 70 min at 4° C. The exosome pellets can then be re-suspended in PBS and subsequently ultra-centrifuged at 120,000×g for another 70 min at 4° C. The exosome pellets can be re-suspended in electroporation buffer.

### 4. Loading of Exosomes with TERT mRNA by Electroporation

Isolated exosomes can be mixed with TERT mRNA in the electroporation buffer, and electroporated at 400 mV and 125 μF capacitance. All exosomes can then be re-suspended in PBS, and ultracentrifuged at 120,000×g for another 70 min at 4° C.

### 5. Systemic (i.v.) Administration of Exosomes into Alzheimer's Disease Subjects

The loaded exosomes can be re-suspended in PBS and then injected intravenously into Alzheimer's disease subjects.

### 6. Characterization of Exosome-Mediated Therapeutic Effects of TERT mRNA Delivery on Alzheimer Pathologies

The AD subjects treated with targeted exosomes loaded with control nucleic acids or TERT mRNA can be periodically assessed for learning and memory tasks. It is contemplated that administration of the therapeutic exosomes will improve the learning and memory of the treated subjects and/or increase clearance of amyloid beta in the subjects' brains.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references disclosed herein, to the extent that they provide exemplary procedural or other details supplementary to those set forth herein, are specifically incorporated herein by reference.

1. A method for generating new neurons in a subject in need thereof, comprising administering a TERT activating therapy to the subject.

2. A method for treating a neurodegenerative disorder in a subject comprising administering a TERT activating therapy to the subject.

3. The method of claim 2, wherein the neurodegenerative disorder comprises Alzheimer's disease.

4. A method for reducing amyloid-β peptide in a subject in need thereof, comprising administering a TERT activating therapy to the subject.

5. A method for treating a premature aging disorder in a subject in need thereof, comprising administering a TERT activating therapy to the subject.

6. The method of claim 5, wherein the premature aging disorder comprises Hutchinson- Gilford progeria syndrome (HGPS), Néstor-Guillermo progeria syndrome, Werner syndrome, Cockayne syndrome, Bloom syndrome, Xeroderma pigmentosum, Ataxia telangiectasia, Trichothiodystrophy, Dyskeratosis congenital, or Mosaic variegated aneuploidy syndrome.

7. The method of any one of claims 1-6, wherein the subject has been diagnosed with the disorder.

8. The method of any one of claims 1-7, wherein the subject has previously been treated for the disorder.

9. The method of claim 8, wherein the subject has been determined to be non-responsive to the previous therapy.

10. The method of any one of claims 1-9, wherein the subject is a human.

11. The method of claim 10, wherein the subject is less than 50 years old.

12. The method of any one of claims 1-11, wherein the method further comprises administration of an additional therapy.

13. The method of any one of claims 1-12, wherein the TERT activating therapy comprises one or more nucleic acids encoding a TERT polypeptide.

14. The method of claim 13, wherein the TERT activating therapy comprises a DNA or RNA encoding a TERT polypeptide to the subject.

15. The method of any one of claims 1-12, wherein the TERT activating therapy comprises a TERT polypeptide.

16. The method of any one of claims 1-15, wherein the TERT activating therapy comprises a nanovesicle comprising a TERT polypeptide or a nucleic acid encoding for a TERT polypeptide.

17. The method of claim 16, wherein the nanovesicle comprises CD47.

18. The method of claim 16 or 17, wherein the nanovesicle comprises a rabies virus glycoprotein peptide.

19. The method of any one of claims 16-18, wherein the nanovesicles are derived from fibroblasts or bone marrow dendritic cells.

20. The method of any one of claims 16-19, wherein the nanovesicles are derived from human cells.

21. The method of any one of claims 1-20, wherein the TERT activating therapy comprises modulation of histone H3K9 methyltransferases (HMTs).

22. The method of claim 21, wherein the modulation comprises repression of the HMT gene or protein.

23. The method of claim 22, wherein the repression comprises genetic silencing of one or more HMT genes.

24. The method of claim 23, wherein the one or more HMT genes comprise one or more of SUV39H1/KMT1A, SUV39H2/KMT1B, SETDB1/KMT1E, SETDB2/KMT1F, PRDM2, G9A/KMT1C, GLP/KMT1D, EHMT1, and RIZ1/KMT8.

25. The method of claim 22, wherein the TERT activating therapy comprises a HMT inhibitor.

26. The method of claim 25, wherein the HMT inhibitor comprises one or more of Chaetocin, BIX-01294, BIX-01338, UNC0638, and BRD4770.

27. The method of any one of claims 1-20, wherein the TERT activating therapy comprises administration of a histone H3K9 demethylase (HDM) polypeptide or a nucleic acid encoding a HDM.

28. The method of claim 27, wherein the HDM polypeptide comprises a polypeptide from one or more of KDM1A/LSD1, KDM3A/JHDM2A, KDM3B/JHDM2B, KDM4A/JHDM3A, KDM4B/JMJD2B, KDM4C/JMJD2C, KDM4D/JMJD2D, KDM7/JHDM1D, and PHF8.

29. The method of any one of claims 1-28, wherein the TERT activating therapy is administered by intravenous injection.

30. The method of any one of claims 1-29, wherein treating comprises one or more of a reduction in amyloid-β peptide, an improvement in learning, an improvement in memory, and the generation of neurons.

31. The method of any one of claims 15-30, wherein the TERT polypeptide comprises a polypeptide with telomerase activity.


SEQUENCE LISTING (14 sequences)

SEQ ID NO: 1 - DNA, 3829 bp, Homo sapiens
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 2 - protein, 1069 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 3 - DNA, 4018 bp, Homo sapiens
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 4 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 5 - DNA, 3826 bp, Mus musculus
gttcccagcctcatctttttcgtcgtggactctcagtggcctgggtcctggctgttttctaagcacacccttgcatcttggttcccgcacgtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaaccctgtctccttccgctagatgaacatgggcattgtagcctcagcactcctggatccacgtcacaagagggactggtcagttgtgaggctaggtcatcctccaaacctctgtgtcatgggtggtatgggagattgtcccagtgccttgtttcctgtaacaggcttgatttctttcctgatgccctcagggaggcagatcctatcccttttagtggcagggatccactagcaccagcacatgaggagtgcacccagtgcacatgggcactgggacagtggacaggtgtgagattcctgggccctggagtcttttcacacctaaccatggagcctgtcccagtacatcagagtgcctcggagatgaaaaaggacatcgagccagtgacctaaattacagcctgaatatactctgaattcatgtgactgccttagctacttctctactgctgtgtagtaaaacaccaagccaacttataaaagcaggattttcctactggagcagcagctgagagtttacatcttgatccataagcacaaaagcacaagacagagagagagagagagagagagagagagagagagagagagagagagagagagagagagagtcagtcagtcagtctaacaaataactaagaaaggtgaagggtgatgaagtccacagggatcacgctagggatgttccatgccttctctgaagctaagattccttggcagcgtttgacagtaaccatagtgggtacctactgagatcactataaagataaaatagggggaagcgtatttgtactgaactggaaaaacatacaaataaagagtaaatcatggaaaaaaaaaaaaaaaaaaa

SEQ ID NO: 6 - protein, 729 aa, Mus musculus
MRPLFQQLLVNHAECQYVRLLRSHCRFRTANQQVTDALNTSPPHLMDLLRLHSSPWQVYGFLRACLCKVVSASLWGTRHNERRFFKNLKKFISLGKYGKLSLQELMWKMKVEDCHWLRSSPGKDRVPAAEHRLRERILATFLFWLMDTYVVQLLRSFFYITESTFQKNRLFFYRKSVWSKLQSIGVRQHLERVRLRELSQEEVRHHQDTWLAMPICRLRFIPKPNGLRPIVNMSYSMGTRALGRRKQAQHFTQRLKTLFSMLNYERTKHPHLMGSSVLGMNDIYRTWRAFVLRVRALDQTPRMYFVKADVTGAYDAIPQGKLVEVVANMIRHSESTYCIRQYAVVRRDSQGQVHKSFRRQVTTLSDLQPYMGQFLKHLQDSDASALRNSVVIEQSISMNESSSSLFDFFLHFLRHSVVKIGDRCYTQCQGIPQGSSLSTLLCSLCFGDMENKLFAEVQRDGLLLRFVDDFLLVTPHLDQAKTFLSTLVHGVPEYGCMINLQKTVVNFPVEPGTLGGAAPYQLPAHCLFPWCGLLLDTQTLEVFCDYSGYAQTSIKTSLTFQSVFKAGKTMRNKLLSVLRLKCHGLFLDLQVNSLQTVCINIYKIFLLQAYRFHACVIQLPFDQRVRKNLTFFLGIISSQASCCYAILKVKNPGMTLKASGSFPPEAAHWLCYQAFLLKLAAHSVIYKCLLGPLRTAQKLLCRKLPEATMTILKAAADPALSTDFQTILD

SEQ ID NO: 7 - DNA, 3633 bp, Mus musculus
gttcccagcctcatctttttcgtcgtggactctcagtggcctgggtcctggctgttttctaagcacacccttgcatcttggttcccgcacgtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcagggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaaccctgtctccttccgctagatgaacatgggcattgtagcctcagcactcctggatccacgtcacaagagggactggtcagttgtgaggctaggtcatcctccaaacctctgtgtcatgggtggtatgggagattgtcccagtgccttgtttcctgtaacaggcttgatttctttcctgatgccctcagggaggcagatcctatcccttttagtggcagggatccactagcaccagcacatgaggagtgcacccagtgcacatgggcactgggacagtggacaggtgtgagattcctgggccctggagtcttttcacacctaaccatggagcctgtcccagtacatcagagtgcctcggagatgaaaaaggacatcgagccagtgacctaaattacagcctgaatatactctgaattcatgtgactgccttagctacttctctactgctgtgtagtaaaacaccaagccaacttataaaagcaggattttcctactggagcagcagctgagagtttacatcttgatccataagcacaaaagcacaagacagagagagagagagagagagagagagagagagagagagagagagagagagagagagagagtcagtcagtcagtctaacaaataactaagaaaggtgaagggtgatgaagtccacagggatcacgctagggatgttccatgccttctctgaagctaagattccttggcagcgtttgacagtaaccatagtgggtacctactgagatcactataaagataaaatagggggaagcgtatttgtactgaactggaaaaacatacaaataaagagtaaatcatggaaaaaaaaaaaaaaaaaaa

SEQ ID NO: 8 - protein, 584 aa, Mus musculus
MDTYVVQLLRSFFYITESTFQKNRLFFYRKSVWSKLQSIGVRQHLERVRLRELSQEEVRHHQDTWLAMPICRLRFIPKPNGLRPIVNMSYSMGTRALGRRKQAQHFTQRLKTLFSMLNYERTKHPHLMGSSVLGMNDIYRTWRAFVLRVRALDQTPRMYFVKADVTGAYDAIPQGKLVEVVANMIRHSESTYCIRQYAVVRRDSQGQVHKSFRRQVTTLSDLQPYMGQFLKHLQDSDASALRNSVVIEQSISMNESSSSLFDFFLHFLRHSVVKIGDRCYTQCQGIPQGSSLSTLLCSLCFGDMENKLFAEVQRDGLLLRFVDDFLLVTPHLDQAKTFLSTLVHGVPEYGCMINLQKTVVNFPVEPGTLGGAAPYQLPAHCLFPWCGLLLDTQTLEVFCDYSGYAQTSIKTSLTFQSVFKAGKTMRNKLLSVLRLKCHGLFLDLQVNSLQTVCINIYKIFLLQAYRFHACVIQLPFDQRVRKNLTFFLGIISSQASCCYAILKVKNPGMTLKASGSFPPEAAHWLCYQAFLLKLAAHSVIYKCLLGPLRTAQKLLCRKLPEATMTILKAAADPALSTDFQTILD

SEQ ID NO: 9 - DNA, 4335 bp, Mus musculus
gttcccagcctcatctttttcgtcgtggactctcagtggcctgggtcctggctgttttctaagcacacccttgcatcttggttcccgcacgtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccggggaaggaccgtgtccccgctgcagagcaccgtctgagggagaggatcctggctacgttcctgttctggctgatggacacatacgtggtacagctgcttaggtcattcttttacatcacagagagcacattccagaagaacaggctcttcttctaccgtaagagtgtgtggagcaagctgcagagcattggagtcaggcaacaccttgagagagtgcggctacgggagctgtcacaagaggaggtcaggcatcaccaggacacctggctagccatgcccatctgcagactgcgcttcatccccaagcccaacggcctgcggcccattgtgaacatgagttatagcatgggtaccagagctttgggcagaaggaagcaggcccagcatttcacccagcgtctcaagactctcttcagcatgctcaactatgagcggacaaaacatcctcaccttatggggtcttctgtactgggtatgaatgacatctacaggacctggcgggcctttgtgctgcgtgtgcgtgctctggaccagacacccaggatgtactttgttaaggcagatgtgaccggggcctatgatgccatcccccagggtaagctggtggaggttgttgccaatatgatcaggcactcggagagcacgtactgtatccgccagtatgcagtggtccggagagatagccaaggccaagtccacaagtcctttaggagacaggtcaccaccctctctgacctccagccatacatgggccagttccttaagcatctgcaggattcagatgccagtgcactgaggaactccgttgtcatcgagcagagcatctctatgaatgagagcagcagcagcctgtttgacttcttcctgcacttcctgcgtcacagtgtcgtaaagattggtgacaggtgctatacgcagtgccagggcatcccccagggctccagcctatccaccctgctctgcagtctgtgtttcggagacatggagaacaagctgtttgctgaggtgcagcgggatgggttgcttttacgttttgttgatgactttctgttggtgacgcctcacttggaccaagcaaaaaccttcctcagcaccctggtccatggcgttcctgagtatgggtgcatgataaacttgcagaagacagtggtgaacttccctgtggagcctggtaccctgggtggtgcagctccataccagctgcctgctcactgcctgtttccctggtgtggcttgctgctggacactcagactttggaggtgttctgtgactactcaggttatgcccagacctcaattaagacgagcctcaccttccagagtgtcttcaaagctgggaagaccatgcggaacaagctcctgtcggtcttgcggttgaagtgtcacggtctatttctagacttgcaggtgaacagcctccagacagtctgcatcaatatatacaagatcttcctgcttcaggcctacaggttccatgcatgtgtgattcagcttccctttgaccagcgtgttaggaagaacctcacattctttctgggcatcatctccagccaagcatcctgctgctatgctatcctgaaggtcaagaatccaggaatgacactaaaggcctctggctcctttcctcctgaagccgcacattggctctgctaccaggccttcctgctcaagctggctgctcattctgtcatctacaaatgtctcctgggacctctgaggacagcccaaaaactgctgtgccggaagctcccagaggcgacaatgaccatccttaaagctgcagctgacccagccctaagcacagactttcagaccattttggactaaccctgtctccttccgctagatgaacatgggcattgtagcctcagcactcctggatccacgtcacaagagggactggtcagttgtgaggctaggtcatcctccaaacctctgtgtcatgggtggtatgggagattgtcccagtgccttgtttcctgtaacaggcttgatttctttcctgatgccctcagggaggcagatcctatcccttttagtggcagggatccactagcaccagcacatgaggagtgcacccagtgcacatgggcactgggacagtggacaggtgtgagattcctgggccctggagtcttttcacacctaaccatggagcctgtcccagtacatcagagtgcctcggagatgaaaaaggacatcgagccagtgacctaaattacagcctgaatatactctgaattcatgtgactgccttagctacttctctactgctgtgtagtaaaacaccaagccaacttataaaagcaggattttcctactggagcagcagctgagagtttacatcttgatccataagcacaaaagcacaagacagagagagagagagagagagagagagagagagagagagagagagagagagagagagagagtcagtcagtcagtctaacaaataactaagaaaggtgaagggtgatgaagtccacagggatcacgctagggatgttccatgccttctctgaagctaagattccttggcagcgtttgacagtaaccatagtgggtacctactgagatcactataaagataaaatagggggaagcgtatttgtactgaactggaaaaacatacaaataaagagtaaatcatggaaaaaaaaaaaaaaaaaaa

SEQ ID NO: 10 - protein, 1122 aa, Mus musculus
MTRAPRCPAVRSLLRSRYREVWPLATFVRRLGPEGRRLVQPGDPKIYRTLVAQCLVCMHWGSQPPPADLSFHQVSSLKELVARVVQRLCERNERNVLAFGFELLNEARGGPPMAFTSSVRSYLPNTVIETLRVSGAWMLLLSRVGDDLLVYLLAHCALYLLVPPSCAYQVCGSPLYQICATTDIWPSVSASYRPTRPVGRNFTNLRFLQQIKSSSRQEAPKPLALPSRGTKRHLSLTSTSVPSAKKARCYPVPRVEEGPHRQVLPTPSGKSWVPSPARSPEVPTAEKDLSSKGKVSDLSLSGSVCCKHKPSSTSLLSPPRQNAFQLRPFIETRHFLYSRGDGQERLNPSFLLSNLQPNLTGARRLVEIIFLGSRPRTSGPLCRTHRLSRRYWQMRPLFQQLLVNHAECQYVRLLRSHCRFRTANQQVTDALNTSPPHLMDLLRLHSSPWQVYGFLRACLCKVVSASLWGTRHNERRFFKNLKKFISLGKYGKLSLQELMWKMKVEDCHWLRSSPGKDRVPAAEHRLRERILATFLFWLMDTYVVQLLRSFFYITESTFQKNRLFFYRKSVWSKLQSIGVRQHLERVRLRELSQEEVRHHQDTWLAMPICRLRFIPKPNGLRPIVNMSYSMGTRALGRRKQAQHFTQRLKTLFSMLNYERTKHPHLMGSSVLGMNDIYRTWRAFVLRVRALDQTPRMYFVKADVTGAYDAIPQGKLVEVVANMIRHSESTYCIRQYAVVRRDSQGQVHKSFRRQVTTLSDLQPYMGQFLKHLQDSDASALRNSVVIEQSISMNESSSSLFDFFLHFLRHSVVKIGDRCYTQCQGIPQGSSLSTLLCSLCFGDMENKLFAEVQRDGLLLRFVDDFLLVTPHLDQAKTFLSTLVHGVPEYGCMINLQKTVVNFPVEPGTLGGAAPYQLPAHCLFPWCGLLLDTQTLEVFCDYSGYAQTSIKTSLTFQSVFKAGKTMRNKLLSVLRLKCHGLFLDLQVNSLQTVCINIYKIFLLQAYRFHACVIQLPFDQRVRKNLTFFLGIISSQASCCYAILKVKNPGMTLKASGSFPPEAAHWLCYQAFLLKLAAHSVIYKCLLGPLRTAQKLLCRKLPEATMTILKAAADPALSTDFQTILD

SEQ ID NO: 11 - protein, 29 aa, Rabies virus
YTIWMPENPRPGTPCDIFTNSRGKRASNG

SEQ ID NO: 12 - protein, 30 aa, Rabies Virus
YTIWMPENPRPGTPCDIFTNSRGKRASNGC

SEQ ID NO: 13 - protein, 34 aa, Rabies virus
YTIWMPENPRPGTPCDIFTNSRGKRASNGGGGGC

SEQ ID NO: 14 - protein, 42 aa, Rabies virus
  MISC_FEATURE (34)..(42): Xaa is D-arginine
YTIWMPENPRPGTPCDIFTNSRGKRASNGGGGGXXXXXXXXX